(19) United States
(12) Patent Application Publication
(10) Pub. No.: US 2026/0060584 A1
(43) Pub. Date: Mar. 5, 2026

(54) ENDOVASCULAR THERAPY DEVICE

(71) Applicant: Covidien LP, Mansfield, MA (US)

(72) Inventors: Dale F. Seeley, Spring Park, MN (US); Robert L. Olson, White Bear Lake, MN (US)

(21) Appl. No.: 19/314,295

(22) Filed: Aug. 29, 2025

Related U.S. Application Data
(60) Provisional application No. 63/689,410, filed on Aug. 30, 2024.

(51) Int. Cl. A61B 5/24

(52) U.S. Cl. CPC A61B 5/24

(57) ABSTRACT

An endovascular device includes a plurality of conductor wires, a lead body, and a plurality of electrodes. The lead body includes at least a first lead body portion, a second lead body portion, and a third lead body portion. Each electrode of the plurality of electrodes is electrically connected to at least one conductor wire of the plurality of conductor wires. The first lead body portion defines a first lead body portion longitudinal axis, the second lead body portion defines a second lead body portion longitudinal axis, and the third lead body portion defines a third lead body portion longitudinal axis. In a first configuration, the second lead body portion longitudinal axis and the third lead body portion longitudinal axis are coaxial. In a second configuration, the second lead body portion longitudinal axis and the third lead body portion longitudinal axis are axially offset.

### CROSS-REFERENCE TO RELATED APPLICATIONS

This application claims the benefit of and priority to U.S. Provisional Patent Application Ser. No. 63/689,410 filed Aug. 30, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes and/or senses one or more patient parameters with the aid of the one or more electrodes.

### SUMMARY

This disclosure describes example endovascular medical devices and systems configured to endovascularly deliver electrical stimulation therapy to a patient (e.g., to one or more nerves or brain targets) and/or sense one or more patient parameters (e.g., nerve signals, brain signals, and/or other physiological parameters), and related methods. In particular, this disclosure describes configurations for structures of medical devices and systems that facilitate delivery of electrical stimulation therapy and/or sensing of patient parameters from an endovascular location.

Medical devices can be configured to electrically connect to electrodes via conductive pathways (e.g., conductor wires) that run along an elongated body of an endovascular device (e.g., a medical lead). The elongated body of the endovascular device can be navigated to a target location within a patient such that the electrodes can be placed proximate the target location. The medical device can be configured to deliver electrical stimulation therapy and/or sense the one or more patient parameters via the electrodes. The medical device can be configured to receive one or more portions of the elongated body to facilitate electrical connection to the electrodes via one or more conductive pathways (e.g., conductor wires). For example, the medical device can include one or more connector portions (e.g., otherwise referred to as receptacles or cavities) that include electrical contacts configured to electrically connect the electrodes of the endovascular device to therapy generation circuitry and/or sensing circuitry via the conductor wires.

In some examples, a medical device can include more than one connector portion such that the medical device is configured to receive multiple elongated bodies (e.g., multiple leads) and/or multiple portions of a single elongated body (e.g., multiple portions of a single lead). Medical devices with multiple connector portions and/or more electrical contacts enables delivery of electrical stimulation therapy and/or sensing via a relatively greater number of electrodes, which can in turn facilitate a greater therapeutic effect of stimulation (e.g., due to greater electrode area coverage), more electrode combinations for selectively targeting tissue of interest, and/or more precise measurements (e.g., in the case of sensing). However, endovascular systems with multiple leads and/or leads with multiple branching portions that connect to different connector portions of the medical device can increase the cross-sectional profile of the system. Systems with multiple leads and/or leads with multiple branching portions may be relatively more difficult to introduce and/or navigate through vasculature of the patient as compared to systems with a single, linear elongated body having a relatively uniform outer cross-sectional dimension. For example, systems with multiple leads and/or leads with multiple branching portions can necessitate the use of relatively larger delivery catheters and/or sheaths. Additionally, delivery catheters and/or sheaths can be relatively more difficult to retract overtop of systems having multiple leads and/or leads with multiple branching portions.

In the examples described herein, an endovascular device (e.g., a medical lead) has multiple body portions (e.g., configured for electrical connection to multiple connector portions of a medical device). In some examples, the endovascular device is configured such that the multiple body portions can transform to a relatively low-profile configuration (e.g., in which at least some of the multiple body portions are coaxial or nearly coaxial) for use with a delivery catheter and/or sheath. For example, the endovascular device can have a first configuration in which the multiple body portions are positioned coaxially (e.g., to facilitate relatively easier introduction into and/or movement relative to a delivery catheter and/or sheath) and a second configuration in which the multiple body portions are not coaxial (e.g., such as to enable electrical connection to multiple respective non-coaxial connector portions of a medical device). Such configurations in which the multiple body portions of the endovascular device are linearly and/or coaxially arranged can facilitate relatively easier movement relative to the delivery catheter and/or sheath (e.g., because the delivery catheter and/or sheath can be more easily retracted from overtop the endovascular device), and/or enable use of a relatively smaller delivery catheter and/or sheath (e.g., having a smaller cross-sectional dimension, such as a diameter). Additionally, by being able to transform to a configuration in which all portions of the elongated body of the endovascular device are linearly and/or coaxially arranged (e.g., such as for use with a sheath and/or delivery catheter), the need for supplemental lead connectors and/or other lead extensions (e.g., which may otherwise facilitate connection to medical devices with multiple connector portions) is advantageously reduced or even eliminated. The endovascular devices described in this disclosure can enable existing medical devices with multiple, non-coaxial connector portions to be programmed for delivering electrical stimulation therapy and/or sensing via electrodes of the endovascular device that are positioned endovascularly.

In some examples herein, an endovascular device includes an elongated lead body and conductor wires extending along (e.g., within) the lead body. In some examples, each of the conductor wires is configured to electrically connect one or more electrodes of the endovascular device to a medical device. The lead body includes a first lead body portion (e.g., which may be a distal lead body portion) including a first group of conductor wires. One or more electrodes can be electrically connected to the conductor wires of the first group of conductor wires. The lead body also includes multiple proximal lead body portions (e.g., for electrical connection to multiple connector portions of a medical device). For example, the lead body can include at least a second lead body portion including a second group of conductor wires and a third lead body portion including a third group of conductor wires. In some examples, the second group of conductor wires is configured to electrically connect to electrical contacts of a first connector portion of the medical device and the third group of conductor wires is configured to electrically connect to electrical contacts of a second connector portion of the medical device.

In some examples herein, the first lead body portion (e.g., which may be a distal body portion) and the associated group of conductor wires splits to form multiple proximal lead body portions and associated groups of conductor wires (e.g., for connection to multiple connector portions of a medical device). For example, in some examples, the lead body includes a bifurcation or junction where the first lead body with first group of conductor wires splits to form the second lead body portion with the second group of conductor wires and the third lead body portion with the third group of conductor wires (e.g., in which the second lead body portion and the third lead body portion are proximal lead body portions configured to be positioned in respective connector portions of a medical device). In some examples, the lead body includes a connection body at the bifurcation or junction that enables the third lead body portion to extend proximally of and coaxial to the second lead body portion (e.g., in a first configuration of the lead body for use with a delivery catheter and/or sheath). In some examples, the connection body can be configured to reversibly deform to accommodate transformation of the lead body to a second configuration in which the second lead body portion and the third lead body portion can be received by respective non-coaxial connector portions of a medical device.

In some examples, the first lead body portion and the associated group of conductor wires transitions to form multiple proximal lead body portions without splitting (e.g., without a bifurcation). For example, in some examples, the lead body includes multiple proximal lead body portions that are configured to bend relative to each other to enable the multiple proximal body portions to be received by respective non-coaxial connector portions of a medical device. In some examples, the medical lead includes one or more extension elements positioned between and connecting two or more proximal lead body portions. For example, in some examples, an extension element is positioned between the second lead body portion and the third lead body portion that enables the third lead body portion to bend relative to the second lead body portion such that the second lead body portion and the third lead body portion can be received by respective non-coaxial connector portions of a medical devoce.

Groups of conductor wires can include suitable arrangements to facilitate mechanical robustness (e.g., fatigue resistance) while also maintaining a relatively low cross-sectional profile. In some examples, conductor wires are arranged in coils (e.g., single wire or multi-wire coils). In some examples, the coils described herein can have suitable arrangements (e.g., pitch, spacing, size, or other suitable coil parameters) to facilitate “splitting” (e.g., bifurcation) of conductor wires (e.g., such that different conductor wires can be routed to non-coaxial connector portions of a medical device or lead extension). In some examples, conductor wires are arranged in sections of uncoiled (e.g., substantially straight or straight) wires. In some examples, uncoiled (e.g., substantially straight or straight) conductor wires are positioned within a multi-wire coil of conductor wires. Such uncoiled within coiled arrangements of conductor wires can facilitate mechanical robustness (e.g., fatigue resistance) of the conductor wires while also maintaining a relatively low cross-sectional profile of the group of conductor wires.

In some examples, an endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires splits to form the second group of conductor wires and the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.

In some examples, an endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires transitions to form the second group of conductor wires and the third group of conductor wires, wherein the third group of conductor wires is configured to extend through the second lead body portion to the third lead body portion, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.

In some examples, an endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.

In some examples, a method includes introducing a medical lead into vasculature of a patient, the medical lead includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires splits to form the second group of conductor wires and the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, an wherein in a second configuration, the second lead body portion longitudinal axis and the third lead body portion longitudinal axis are axially offset; and advancing the medical lead until the plurality of electrodes are adjacent a target location in the vasculature of the patient.

In some examples, a method includes introducing a medical lead into vasculature of a patient, the medical lead includes a plurality of conductor wires including at least a first group of conductor wires, a second group of conductor wires, and a third group of conductor wires, a lead body including a first lead body portion, a second lead body portion, and a third lead body portion, and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires transitions to form the second group of conductor wires and the third group of conductor wires, wherein the third group of conductor wires is configured to extend through the second lead body portion to the third lead body portion, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset; and advancing the medical lead until the plurality of electrodes are adjacent a target location in the vasculature of the patient.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example therapy system including an endovascular device configured to deliver electrical stimulation therapy to a target tissue site of a patient and/or sense a patient parameter from an endovascular location.

FIG. 2 is a functional block diagram illustrating components of an example medical device of the therapy system of FIG. 1.

FIG. 3A illustrates an example endovascular therapy system including an endovascular device that includes a first lead body portion that splits to form a second lead body portion and a third lead body portion.

FIG. 3B illustrates a portion of the example endovascular therapy system of FIG. 3A.

FIG. 3C illustrates a portion of the example endovascular therapy system of FIG. 3A.

FIG. 3D illustrates an example connection body from the example endovascular therapy systems of FIG. 3B and FIG. 3C.

FIG. 4 illustrates a first group of conductor wires that splits to form a second group of conductor wires and a third group of conductor wires.

FIG. 5 illustrates a first group of conductor wires that splits to form a second group of conductor wires and a third group of conductor wires.

FIG. 6A illustrates an example endovascular therapy system including an endovascular device that includes a first lead body portion that transitions to form a second lead body portion and a third lead body portion.

FIG. 6B illustrates a portion of the example endovascular therapy system of FIG. 6A.

FIG. 6C illustrates a portion of the example endovascular therapy system of FIG. 6A.

FIG. 6D illustrates a cross-sectional view of a portion of the example endovascular therapy system of FIG. 6C.

FIG. 7 illustrates an example medical device with multiple connector portions.

FIG. 8 illustrates an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, and the like) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson's disease, dystonia, or cerebellar outflow tremor.

While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

Medical devices can be configured to electrically connect to electrodes via conductive pathways (e.g., conductor wires) that run along an elongated body of an endovascular device (e.g., a medical lead). The elongated body of the endovascular device can be navigated to a target location within a patient such that the electrodes can be placed proximate the target location. The medical device can be configured to deliver electrical stimulation therapy and/or sense the one or more patient parameters via the electrodes. The medical device can be configured to receive one or more portions of the elongated body to facilitate electrical connection to the electrodes via one or more conductive pathways (e.g., conductor wires). For example, the medical device can include one or more connector portions (e.g., otherwise referred to as receptacles or cavities) that include electrical contacts configured to electrically connect the electrodes of the endovascular device to therapy generation circuitry and/or sensing circuitry via the conductor wires.

In some examples, a medical device can include more than one connector portion such that the medical device is configured to receive multiple elongated bodies (e.g., multiple leads) and/or multiple portions of a single elongated body (e.g., multiple portions of a single lead). Medical devices with multiple connector portions and/or more electrical contacts enables delivery of electrical stimulation therapy and/or sensing via a relatively greater number of electrodes, which can in turn facilitate a greater therapeutic effect of stimulation (e.g., due to greater electrode area coverage), more electrode combinations for selectively targeting tissue of interest, and/or more precise measurements (e.g., in the case of sensing). However, endovascular systems with multiple leads and/or leads with multiple branching portions that connect to different connector portions of the medical device can increase the cross-sectional profile of the system. Systems with multiple leads and/or leads with multiple branching portions may be relatively more difficult to introduce and/or navigate through vasculature of the patient as compared to systems with a single, linear elongated body having a relatively uniform outer cross-sectional dimension. For example, systems with multiple leads and/or leads with multiple branching portions can necessitate the use of relatively larger delivery catheters and/or sheaths. Additionally, delivery catheters and/or sheaths can be relatively more difficult to retract overtop of systems having multiple leads and/or leads with multiple branching portions.

In the examples described herein, an endovascular device (e.g., a medical lead) has multiple body portions (e.g., configured for electrical connection to multiple connector portions of a medical device). In some examples, the endovascular device is configured such that the multiple body portions can transform to a relatively low-profile configuration (e.g., in which at least some of the multiple body portions are coaxial or nearly coaxial) for use with a delivery catheter and/or sheath. For example, the endovascular device can have a first configuration in which the multiple body portions are positioned coaxially (e.g., to facilitate relatively easier introduction into and/or movement relative to a delivery catheter and/or sheath) and a second configuration in which the multiple body portions are not coaxial (e.g., such as to enable electrical connection to multiple respective non-coaxial connector portions of a medical device). Such configurations in which the multiple body portions of the endovascular device are linearly and/or coaxial arranged can facilitate relatively easier movement relative to the delivery catheter and/or sheath (e.g., because the delivery catheter and/or sheath can be more easily retracted overtop the endovascular device), and/or enable use of a relatively smaller delivery catheter and/or sheath (e.g., having a smaller cross-sectional dimension, such as a diameter). Additionally, by being able to transform to a configuration in which all portions of the elongated body of the endovascular device are linearly and/or coaxially arranged (e.g., such as for use with a sheath and/or delivery catheter), the need for supplemental lead connectors and/or other lead extensions (e.g., which may otherwise facilitate connection to medical devices with multiple connector portions) is advantageously reduced or even eliminated. The endovascular devices described in this disclosure can enable existing medical devices with multiple, non-coaxial connector portions to be programmed for delivering electrical stimulation therapy and/or sensing via electrodes of the endovascular device that are positioned endovascularly.

In some examples herein, an endovascular device includes an elongated lead body and conductor wires extending along (e.g., within) the lead body. In some examples, each of the conductor wires is configured to electrically connect one or more electrodes of the endovascular device to a medical device. The lead body includes a first lead body portion (e.g., which may be a distal lead body portion) including a first group of conductor wires. One or more electrodes can be electrically connected to the conductor wires of the first group of conductor wires. The lead body also includes multiple proximal lead body portions (e.g., for electrical connection to multiple connector portions of a medical device). For example, the lead body can include at least a second lead body portion including a second group of conductor wires and a third lead body portion including a third group of conductor wires. In some examples, the second group of conductor wires is configured to electrically connect to electrical contacts of a first connector portion of the medical device and the third group of conductor wires is configured to electrically connect to electrical contacts of a second connector portion of the medical device.

In some examples herein, the first lead body portion (e.g., which may be a distal body portion) and the associated group of conductor wires splits to form multiple proximal lead body portions and associated groups of conductor wires (e.g., for connection to multiple connector portions of a medical device). For example, in some examples, the lead body includes a bifurcation or junction where the first lead body with first group of conductor wires splits to form the second lead body portion with the second group of conductor wires and the third lead body portion with the third group of conductor wires (e.g., in which the second lead body portion and the third lead body portion are proximal lead body portions configured to be positioned in respective connector portions of a medical device). In some examples, the lead body includes a connection body at the bifurcation or junction that enables the third lead body portion to extend proximally of and coaxial to the second lead body portion (e.g., in a first configuration of the lead body for use with a delivery catheter and/or sheath). In some examples, the connection body can be configured to reversibly deform to accommodate transformation of the lead body to a second configuration in which the second lead body portion and the third lead body portion can be received by respective non-coaxial connector portions of a medical device.

In some examples, the first lead body portion and the associated group of conductor wires transitions to form multiple proximal lead body portions without splitting (e.g., without a bifurcation). For example, in some examples, the lead body includes multiple proximal lead body portions that are configured to bend relative to each other to enable the multiple proximal body portions to be received by respective non-coaxial connector portions of a medical device. In some examples, the medical lead includes one or more extension elements positioned between and connecting two or more proximal lead body portions. For example, in some examples, an extension element is positioned between the second lead body portion and the third lead body portion that enables the third lead body portion to bend relative to the second lead body portion such that the second lead body portion and the third lead body portion can be received by respective non-coaxial connector portions of a medical device.

Groups of conductor wires can include suitable arrangements to facilitate mechanical robustness (e.g., fatigue resistance) while also maintaining a relatively low cross-sectional profile. In some examples, conductor wires are arranged in coils (e.g., single wire or multi-wire coils). In some examples, the coils described herein can have suitable arrangements (e.g., pitch, spacing, size, or other suitable coil parameters) to facilitate “splitting” (e.g., bifurcation) of conductor wires (e.g., such that different conductor wires can be routed to non-coaxial connector portions of a medical device or lead extension). In some examples, conductor wires are arranged in sections of uncoiled (e.g., substantially straight or straight) wires. In some examples, uncoiled (e.g., substantially straight or straight) conductor wires are positioned within a multi-wire coil of conductor wires. Such uncoiled within coiled arrangements of conductor wires can facilitate mechanical robustness (e.g., fatigue resistance) of the conductor wires while also maintaining a relatively low cross-sectional profile of the group of conductor wires.

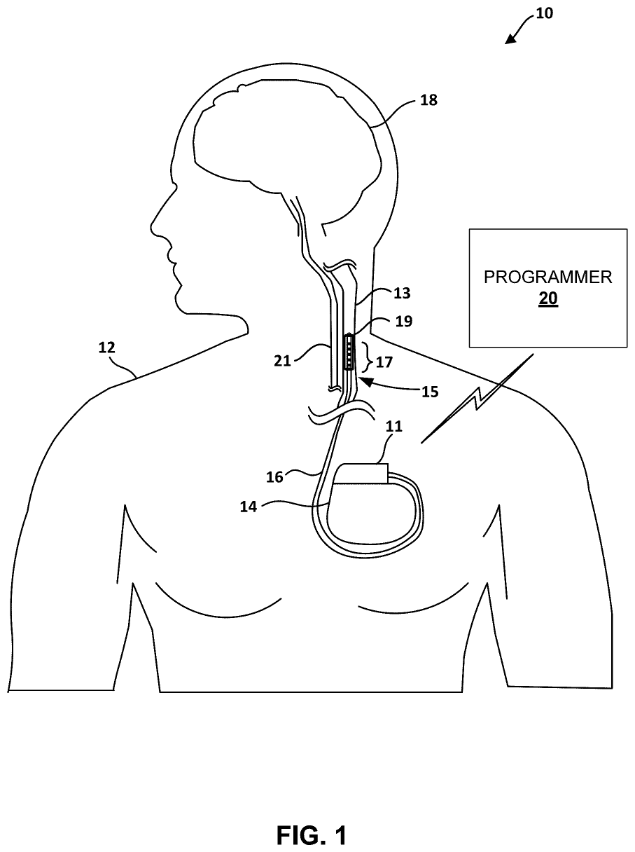

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example shown in FIG. 1, medical device 14 is configured to deliver electrical stimulation therapy (e.g., VNS) to a vagus nerve 21 of patient 12 and/or sense bioelectrical signals via electrodes 17. However, in other examples, therapy system 10 and/or medical device 14 is configured to deliver electrical stimulation therapy to other target sites in patient 12, such as, but not limited to brain 18 of patient 12 and/or sense bioelectrical brain signals in brain 18 via electrodes 17.

In the example shown in FIG. 1, endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrodes 17 in apposition with a vessel wall (e.g., of jugular vein 13). In some examples, as discussed in relation to later examples, expandable structure 19 is mechanically coupled (e.g., directly mechanically coupled) to a portion of endovascular device 16 via a suitable mechanical connection (e.g., welding, crimped connection, or the like). In other examples, expandable structure 19 is proximal to a distal end of endovascular device 16. Medical device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrodes 17. For example, endovascular device 16 can be a medical lead, a catheter, a guidewire, or another elongated body carrying electrodes 17 and configured to be electrically coupled to medical device 14 via an electrically conductive pathway (e.g., via one or more conductor wires) that runs between medical device 14 and electrodes 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. However, other access points may be used to introduce endovascular device 16 into vasculature of a patient, such as, but not limited to, a radial artery.

As used herein, “about” may indicate the exact value or nearly the exact value to the extent permitted by manufacturing tolerances. “About” can also refer to a certain percentage of the recited value (e.g., within about 1%, 5%, or 10%).

Endovascular device 16 is configured to be introduced in the vasculature of patient 12, such as to access jugular vein 13 and/or relatively more distal locations in a patient, such as the middle cerebral artery (MCA) in a brain of a patient. Endovascular device 16 may include an elongated body that is structurally configured to be relatively flexible, pushable, and relatively kink- and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands) proximate electrodes 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one of endovascular device 16 is introduced into, positioned in, and/or implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16, including electrodes 17 and/or expandable structure 19, can be positioned in and/or implanted within a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods (otherwise referred to as screening periods), such as to determine, evaluate, or confirm an efficacy of stimulation and/or sensing, and/or to select electrical stimulation parameters for chronic therapy delivery.

The electrical stimulation therapy described herein (e.g., VNS, DBS, or the like) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, obesity, movement disorders, other neurodegenerative impairment, seizure disorders, psychiatric disorders (e.g., mood disorders). Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, one or more electrodes 17 are positioned on (e.g., mechanically coupled to, defined by, or otherwise carried by) expandable structure 19 of endovascular device 16, which is configured to expand radially outwards from a relatively low-profile (e.g., radially compressed) delivery configuration to a deployed configuration. This may enable electrodes 17 to be held in apposition with a blood vessel wall, promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example shown in FIG. 1, endovascular device 16 is directly or indirectly mechanically and electrically coupled to medical device 14 via a header 11 of medical device 14, which defines a plurality of electrical contacts in one or more connector portions (e.g., that are configured to electrically couple electrodes 17 to electrical stimulation generation circuitry and/or sensing circuitry within medical device 14). In some examples, as described in connection with later examples, header 11 and/or another portion of medical device 14 can have multiple connector portions with respective electrical contacts, the multiple connector portions configured to receive and electrically connect to multiple of endovascular device 16 and/or multiple proximal portions of a single one of endovascular device 16.

In some examples, therapy system 10 includes one or more conductor wires (not shown in FIG. 1) extending between medical device 14 and electrodes 17, the one or more conductor wires are configured to carry electrical signals between medical device and electrodes 17 or vice versa. The conductor wires may extend along, be a part of, incorporated into, and/or integrally formed as part of endovascular device 16. In some examples, header 11 includes multiple connector portions, which may be respectively configured to receive one of multiple portions of endovascular device 16. Header 11 may also be referred to as a connector block or connector of medical device 14. Endovascular device 16 may be mechanically coupled and/or electrically coupled to header 11 with the aid of a lead extension. However, in some examples, a lead extension is not used between header 11 and endovascular device 16, and endovascular device is directly mechanically and/or electrically connected to medical device 14 via header 11. In some examples, a trialing cable with multiple connector portions connects electrodes to medical device 14.

In some examples, medical device 14 is configured to be positioned in (e.g., implanted in) patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As shown in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include a combination of activated electrodes (also referred to herein as an electrode combination), a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more of endovascular devices 16 mechanically coupled and/or electrically coupled to medical device 14. The electrode combination may also refer to the polarities of the electrodes in the selected subset. By selecting particular electrode combinations, a user may target particular tissue sites (e.g., anatomic structures) within patient 12 and/or avoid particular tissue sites. In addition, by selecting values for other electrical stimulation parameter values, such as slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may be configured to communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

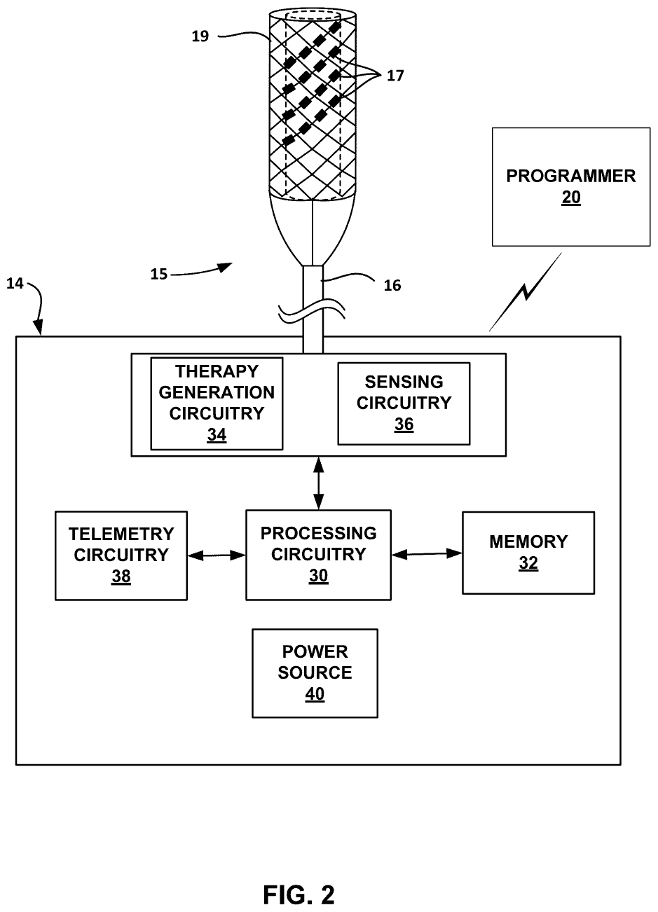

In some examples, in addition to or instead of delivering electrical stimulation to a target location (e.g., vagus nerve 21), medical device 14 or another device is configured to sense one or more patient parameters, such as bioelectric signals, either using electrodes 17 or other types of sensors that are carried by endovascular device 16. Bioelectric signals (also referred to herein as bioelectrical signals) can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19. In some examples, one or more sensors that are different from electrodes 17 are positioned on the same expandable structure (e.g., expandable structure 19) as one or more electrodes 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. Positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils, barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables expandable structure 19 to assume a predetermined shape in the absence of a force (e.g., a compressive or tensile force) holding expandable structure 19 in a relatively low-profile delivery configuration. For example, expandable structure 19 can be configured to expand (e.g., self-expand) radially outwards upon deployment from an outer sheath (e.g., an outer catheter), or upon the proximal withdrawal of a straightening element (e.g., a guidewire, obturator, or a mandrel) positioned in an inner lumen of the endovascular device 16. In some examples, expandable structure 19 is configured to expand radially outwards in response to proximal movement of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 includes a plurality of interconnected struts to form a structure configured to expand radially outward (e.g., from a central longitudinal axis of expandable structure 19). For example, expandable structure 19 can include a tubular member, a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or mechanically coupled together) or can be a unitary structure (e.g., a laser cut nitinol tube). In some examples, expandable structure 19 is referred to herein as having a stent-like structure.

Expandable structure 19 can be mechanically coupled to a portion of endovascular device 16 (e.g., distal portion 15 of endovascular device 16). In some examples, expandable structure 19 is mechanically coupled to endovascular device 16 via a welded connection, a crimped connection, a bonded connection (e.g., via an adhesive and/or another suitable bonding agent), or another suitable mechanical connection.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 (as well as electrodes 17, medical device 14, processing circuitry, etc.) may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration as compared to a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more of endovascular devices may be used for multiple trial periods (e.g., successive trial periods) for determining an efficacy of one or more stimulation parameters and/or one or more sensing parameters.

Therapy system 10 may have any suitable configuration for delivering electrical stimulation to a target tissue site in patient 12 or sensing a patient parameter from an endovascular location (e.g., jugular vein 13). In some examples, therapy system 10 includes a first subset of electrodes of electrodes 17 configured for delivering electrical stimulation therapy and a second subset of electrodes of electrodes 17 configured to for sensing one or more patient parameters. In some examples, some or all electrodes of electrodes 17 are configured for both electrical stimulation therapy and for sensing one or more patient parameters. Therapy system 10 can include any suitable number of electrodes 17 and/or combination of different kinds of electrodes. In some examples, electrodes 17 include electrodes formed via one or more manufacturing processes. For example, electrodes 17 can include a first electrode type (e.g., an electrode configured for delivery of electrical stimulation therapy), a second electrode type (e.g., an electrode configured to sensing a signal), or any suitable combination thereof.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectrical signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger).

In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 38 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of medical device 14 shown in FIG. 2.

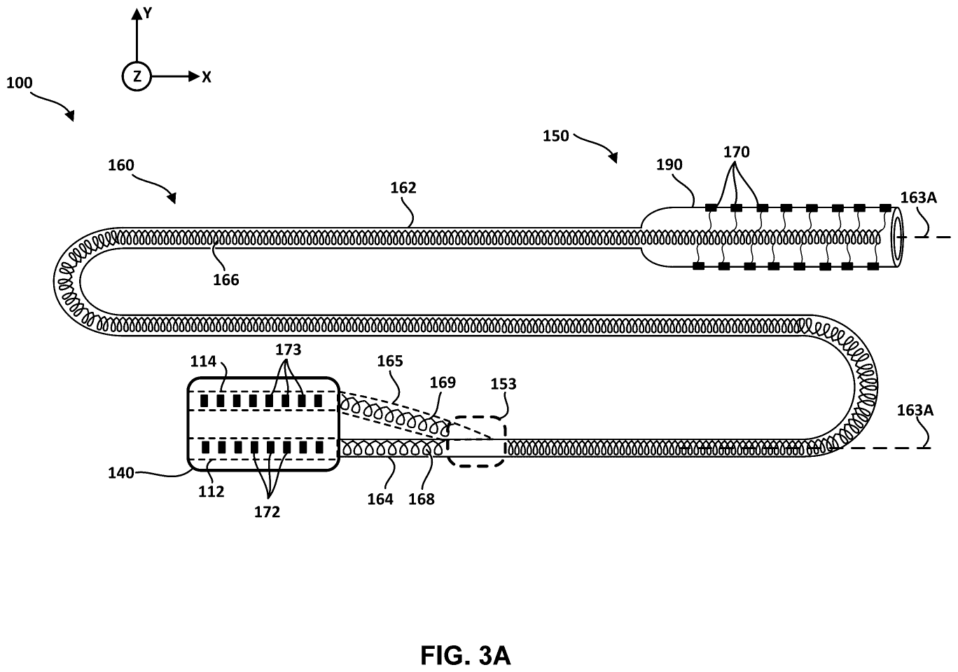

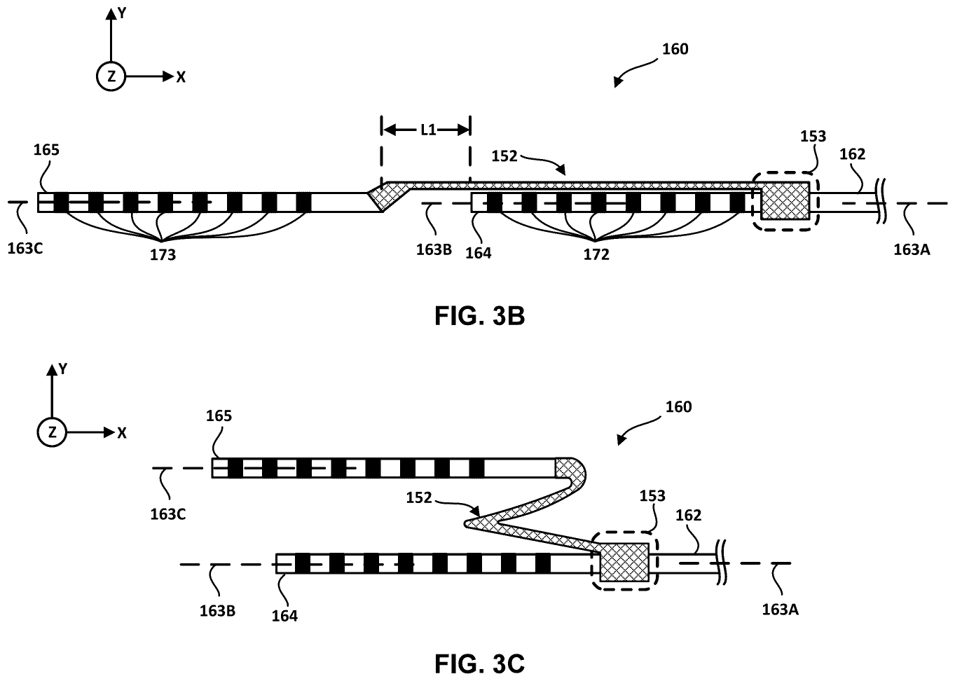

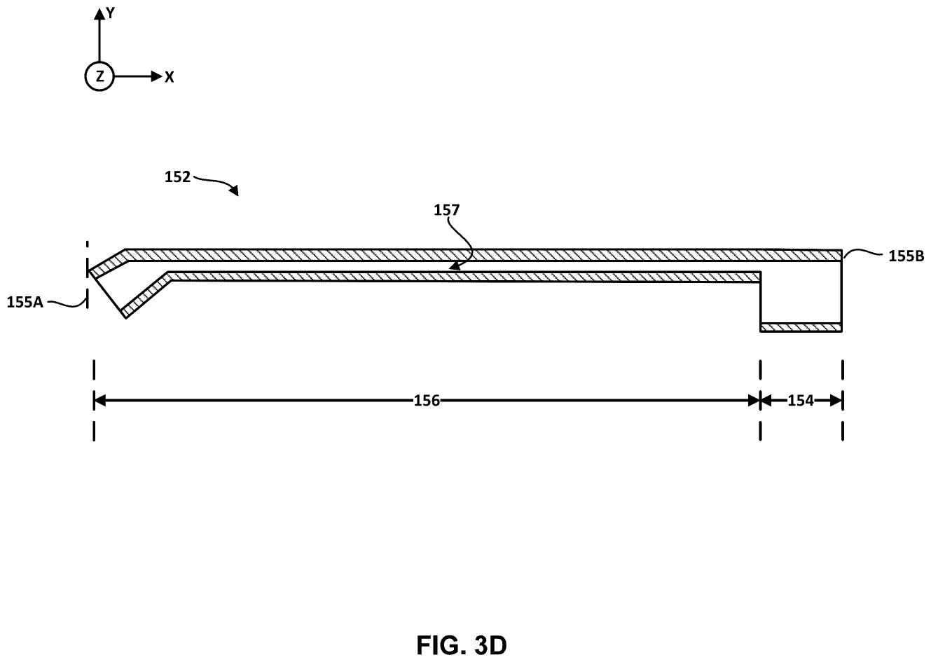

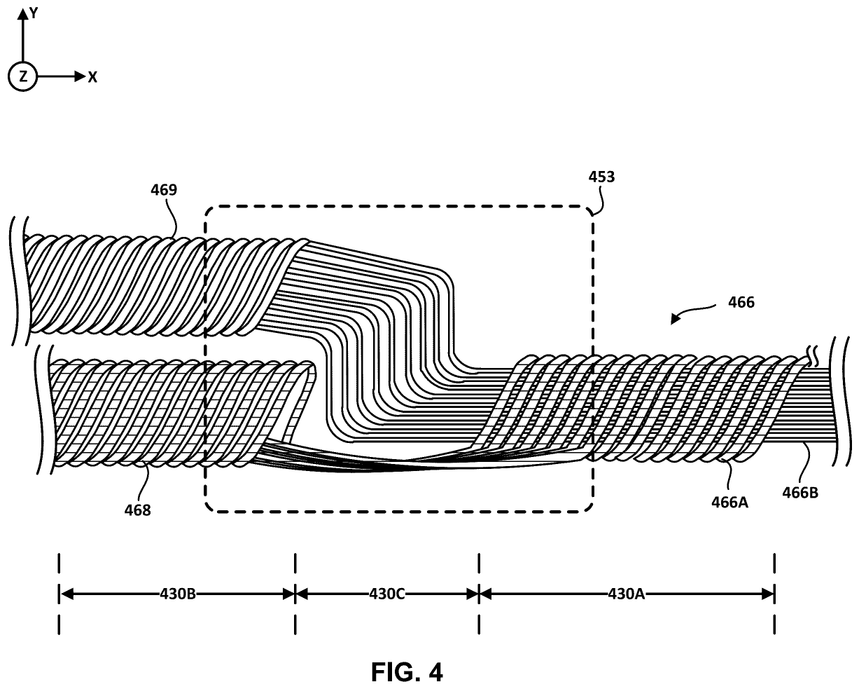

FIG. 3A, FIG. 3B, FIG. 3C, and FIG. 3D illustrate an example endovascular therapy system 100 and various components thereof. Endovascular therapy system 100 is an example of therapy system 10 of FIG. 1. Endovascular therapy system 100 includes a medical device 140, a medical lead 160, and a plurality of electrodes 170 (which are examples of medical device 14, endovascular device 16, and electrodes 17, respectively, as shown and described in connection with FIG. 1, respectively). In some examples, medical device 140 is configured to receive one or more portions of medical lead 160 (e.g., one or more body portions of medical lead 160), and includes another cable extending to circuitry (e.g., processing circuitry, therapy generation circuitry, sensing circuitry, and the like), such as in the case of a lead extension.

In the example of FIG. 3A, medical lead 160 includes a first lead body portion 162, a second lead body portion 164, and a third lead body portion 165 (e.g., where first lead body portion 162, second lead body portion 164, and third lead body portion 165 collectively form a lead body of medical lead 160). In some examples, first lead body portion 162 is a distal portion (e.g., a distal-most portion) of medical lead 160. In some examples, second lead body portion 164 and third lead body portion 165 together form a proximal portion (e.g., a proximal-most portion) of medical lead 160. In some examples, as shown in the examples of FIG. 3A, FIG. 3B, and FIG. 3C, second lead body portion 164 is an integral extension of (e.g., integrally formed with) first lead body portion 162 (e.g., such that first lead body portion 162 transitions to form second lead body portion 164 at a junction 153). In other examples, first lead body portion 162 is formed separately from and mechanically coupled to second lead body portion 164 and third lead body portion 165 at junction 153 (e.g., where junction 153 is at the interface of first lead body portion 162, second lead body portion 164, and third lead body portion 165).

In some examples, as illustrated in FIG. 3A, medical lead 160 includes an expandable structure 190 at a distal portion 150 of medical lead 160 (e.g., a distal portion of first lead body portion 162) to position and/or orient electrodes 170 within vasculature of a patient. In some examples, electrodes 170 are carried by, disposed on, and/or defined by expandable structure 190, which is configured to transform from a relatively low-profile delivery configuration to a deployed configuration in a blood vessel of a patient (e.g., within jugular vein 13 or in a blood vessel in brain 18 of patient 12 as discussed in relation to FIG. 1). Expandable structure 190 is an example of expandable structure 19 discussed in connection with FIG. 1, and can include any suitable shape and materials. In some examples, expandable structure 190 includes a frame with one or more interconnected struts (e.g., including a self-expanding stent comprising a shape-memory material), a balloon, a self-expanding coil, or another suitable expandable structure as described herein.

As shown in the example of FIG. 3A, as well as FIG. 3B and FIG. 3C, first lead body portion 162 portion defines a first lead body portion longitudinal axis 163A, which may be a central longitudinal axis extending through a radial center of first lead body portion 162. As shown in the examples of FIG. 3B and FIG. 3C, second lead body portion 164 defines a second lead body portion longitudinal axis 163B, which may be a central longitudinal axis extending through a radial center of second lead body portion 164, and the third lead body portion 165 defines a third lead body portion longitudinal axis 163C, which may be a central longitudinal axis extending through a radial center of third lead body portion 165. Each of first lead body portion longitudinal axis 163A, second lead body portion longitudinal axis 163B, and third lead body portion longitudinal axis 163C can be aligned (e.g., axially aligned, or coaxial), or offset (e.g., axially offset, which can include parallel) depending on the configuration of medical lead 160.

Medical lead 160 includes a plurality of conductor wires 166, 168, 169 (shown individually as a first group of conductor wires 166, a second group of conductor wires 168, and a third group of conductor wires 169, but collectively referred to herein as plurality of conductor wires 166, 168, 169). In the example of FIG. 3A, first lead body portion 162 includes (e.g., is configured to receive and/or otherwise incorporates) first group of conductor wires 166, second lead body portion 164 includes (e.g., is configured to receive and/or otherwise incorporates) second group of conductor wires 168, and third lead body portion 165 includes (e.g., is configured to receive and/or otherwise incorporates) third group of conductor wires 169.

As shown in the example of FIG. 3A, each of electrodes 170 is electrically connected to at least one conductor wire of first group of conductor wires 166. In some examples, as illustrated in the FIG. 3A, first group of conductor wires 166 splits to form second group of conductor wires 168 and third group of conductor wires 169 at junction 153. This splitting may facilitate connection of second group of conductor wires 168 and third group of conductor wires 169 to separate connector portions of medical device 140. Although the term “split” is used throughout this disclosure to describe the bifurcation (or other division, e.g., trifurcation in some examples) of first group of conductor wires 166 into second group of conductor wires 168 and third group of conductor wires 169, second group of conductor wires 168 and third group of conductor wires 169 can be continuous extensions of the respective subsets of wires that form first group of conductor wires 166. For example, each wire of second group of conductor wires 168 can be a continuous extension of one wire of a first subset of first group of conductor wires 166 and each wire of third group of conductor wires 169 can be a continuous extension of one wire of a second subset of first group of conductor wires 166. In some examples, each of the plurality of conductor wires 166, 168, 169 extend continuously between electrodes 170 and medical device 140 (e.g., such as to electrically connect each of electrodes 170 to circuitry of medical device 140).

In some examples, at least a portion of second lead body portion 164 and/or third lead body portion 165 are configured to be positioned outside of the vasculature to facilitate electrical connection of the plurality of conductor wires 166, 168, 169 to medical device 140. Medical device 140 may be placed in an extravascular portion of the chest or external to patient 12. In some examples, such as in instances of temporary electrical stimulation therapy, sensing, and/or screening, medical device 140 is configured to be positioned externally of patient 12.

In some examples, medical device 140 is configured to receive one or more portions of medical lead 160. For example, medical device 140 can be configured to receive one or more portions of medical lead 160 for electrically connecting circuitry of medical device 140 (e.g., processing circuitry, therapy generation circuitry, and/or sensing circuitry, as described in connection with FIG. 2) to plurality of conductor wires 166, 168, 169. As shown in the example of FIG. 3A, medical device 140 includes a first connector portion 112 and a second connector portion 114. However, medical device 140 can include any suitable number of connector portions (e.g., one connector portion, two connector portions, three connector portions, four connector portions, or more). In some examples, first connector portion 112 is configured to receive a first portion of medical lead 160 (e.g., second lead body portion 164 of medical lead 160) and second connector portion 114 is configured to receive a second portion of medical lead 160 (e.g., third lead body portion 165 of medical lead 160).

In the example of FIG. 3A, second group of conductor wires 168 is configured to electrically connect to medical device 140 via first connector portion 112, and third group of conductor wires 169 is configured to electrically connect to medical device 140 via second connector portion 114. In the example of FIG. 3A, second lead body portion 164 of medical lead 160 includes a first group of electrical contacts 172 that are electrically connected to second group of conductor wires 168 and facilitate electrical connection of at least some electrodes 170 to medical device 140 via first connector portion 112. In some examples, each of first group of electrical contacts 172 is electrically connected to a respective one of second group of conductor wires 168. As shown in the example of FIG. 3B and FIG. 3C, first group of electrical contacts 172 are spaced apart along second lead body portion longitudinal axis 163B. In some examples, first group of electrical contacts 172 are disposed on a proximal portion (e.g., a proximal-most portion) of second lead body portion 164.

In some examples, medical lead 160 includes a second group of electrical contacts 173 that are electrically connected to third group of conductor wires 169 and facilitate electrical connection of at least some electrodes 170 to medical device 140 via second connector portion 114. In some examples, each of second group of electrical contacts 173 is electrically connected to a respective one of third group of conductor wires 169. As shown in the example of FIG. 3B and FIG. 3C, second group of electrical contacts 173 are spaced apart along third lead body portion longitudinal axis 163C. In some examples, second group of electrical contacts 173 are disposed on a proximal portion (e.g., a proximal-most portion) of third lead body portion 165.

In some examples, first group of electrical contacts 172 and second group of electrical contacts 173 are configured to engage respective features (e.g., corresponding electrical contacts) of first connector portion 112 and second connector portion 114, such as for electrically connecting second group of conductor wires 168 and third group of conductor wires 169 to circuitry (e.g., processing circuitry, therapy generation circuitry, sensing circuitry, as discussed in relation to FIG. 2) of medical device 140. For example, in some examples, second lead body portion 164 is configured to be received in first connector portion 112 to electrically connect second group of conductor wires 168 to a first set of electrical contacts of medical device 140 (not shown in FIG. 3A). In some examples, third lead body portion 165 is configured to be received in second connector portion 114 of the medical device 140 to electrically connect third group of conductor wires 169 to a second set of electrical contacts of medical device 140 (not shown in FIG. 3A). In this way, second group of conductor wires 168 is configured to electrically connect to medical device 140 via first group of electrical contacts 172 and third group of conductor wires 169 is configured to electrically connect to medical device 140 via second group of electrical contacts 173.

In some examples, second lead body portion 164 and/or third lead body portion 165 are configured to mechanically couple to medical device 140. In some examples, medical device 140 is configured to receive portions of second lead body portion 164 and/or third lead body portion 165 and/or retain portions of second lead body portion 164 and/or third lead body portion 165 within medical device 140. For example, medical device 140 can include a header (e.g., similar to header 11 in the example of FIG. 1) configured to receive and/or retain portions of second lead body portion 164 and/or third lead body portion 165. In some examples, second lead body portion 164, third lead body portion 165 and/or medical device 140 include features (e.g., mating features) to facilitate mechanical coupling (e.g., snap fit or interference fit features). In some examples, medical device 140 includes one or set screws for mechanically coupling second lead body portion 164 and/or third lead body portion 165 to medical device 140. Second lead body portion 164, third lead body portion 165 and/or medical device 140 can include any suitable features for mechanical connection, including Bal Seal® connectors, spring connectors, push fittings, or other suitable connectors.

As illustrated in the examples of FIG. 3A, FIG. 3B, and FIG. 3C, medical lead 160 is configured to transform between at least a first configuration and second configuration. In the first configuration of medical lead 160 as illustrated in FIG. 3B, medical lead 160 is configured to slide and/or move relative to (e.g., within) a delivery catheter and/or sheath (e.g., in which a delivery catheter and/or sheath radially surrounds medical lead 160 and is retracted overtop of medical lead 160), at least because portions of medical lead 160 are arranged linearly and/or coaxially. In some examples, in the first configuration of medical lead 160, a substantial portion of medical lead 160 (e.g., including at least second lead body portion 164, third lead body portion 165, and a majority of first lead body portion 162) defines a substantially uniform (e.g., uniform or nearly uniform to the extent permitted by manufacturing tolerances) outer cross-sectional dimension (e.g., an outer diameter), which can facilitate relatively easier movement of medical lead 160 within a delivery catheter and/or sheath.

In the second configuration of medical lead 160, as illustrated in FIG. 3A and FIG. 3C, portions of medical lead 160 (e.g., second lead body portion 164 and third lead body portion 165) are configured to mechanically and/or electrically couple to respective, non-coaxial connector portions (e.g., first connector portion 112 and second connector portion 114) of medical device 140. This ability of medical lead 160 to transform between such first configuration and second configuration can enable medical lead 160 to move (e.g., slide) more easily relative to and/or within a lumen of a delivery catheter and/or sheath (e.g., as compared to other medical leads that do not have a configuration in which all or a majority of portions of the medical lead are able to be linearly and/or coaxially arranged), while also enabling medical lead 160 to electrically and/or mechanically couple with multi-connector portion medical devices (e.g., such as medical device 140) configured to receive multiple, non-coaxial portions. Additionally, such configurations of medical lead 160 can advantageously reduce or even eliminate the need for supplemental lead connectors and/or other lead extensions that would otherwise be needed for medical lead 160 to mechanically and/or electrically connect to medical device 140.

The example of FIG. 3B illustrates a portion of medical lead 160 in which medical lead 160 is in the first configuration. In the first configuration, second lead body portion 164 and third lead body portion 165 are coaxial (e.g., second lead body portion 164 and third lead body portion 165 share a common central longitudinal axis) or at least substantially coaxial. For example, as shown in FIG. 3B, second lead body portion longitudinal axis 163B and third lead body portion longitudinal axis 163C are coaxial (e.g., are aligned such that they can be considered a single axis). In some examples, as shown in FIG. 3B, in the first configuration, second lead body portion longitudinal axis 163B and third lead body portion longitudinal axis 163C are also coaxial with first lead body portion longitudinal axis 163A (e.g., such that all lead body portions of medical lead 160 share a common central longitudinal axis).

In some examples, when medical lead 160 is in the first configuration, at least second lead body portion 164 and third lead body portion 165 are configured to pass through a lumen of the delivery catheter. In such examples, in the first configuration, second lead body portion 164 and third lead body portion 165 are configured to enable a delivery catheter (not shown in FIG. 3B) to be retracted over each second lead body portion 164 and third lead body portion 165 in sequence. Such ability of medical lead 160 to transform to the first configuration illustrated in FIG. 3B in which at least second lead body portion 164 and third lead body portion 165 are coaxial and/or are positioned in linear succussion can facilitate relatively easier movement of medical lead 160 relative to and/or within a lumen of the delivery catheter and/or sheath (e.g., because the delivery catheter and/or sheath can be more easily retracted from overtop the endovascular device), and/or enable use of a relatively smaller delivery catheter and/or sheath (e.g., having a smaller cross-sectional dimension, such as a smaller diameter).

In some examples, as shown in the example of FIG. 3B in which medical lead 160 is in the first configuration, all of second group of electrical contacts 173 of third lead body portion 165 are proximal to all electrical contacts of first group of electrical contacts 172 of second lead body portion 164. In this way, second lead body portion 164 and third lead body portion 165 of medical lead 160 can be positioned in sequence (e.g., in sequence along the x-axis direction according to the orthogonal x-y-z axes shown in FIG. 3B), which can facilitate relatively easier movement of medical lead 160 relative to a delivery catheter and/or sheath.

The examples of FIG. 3A and FIG. 3C illustrate a portion of medical lead 160 in which medical lead 160 is in the second configuration. As shown in the example of FIG. 3A, in the second configuration of medical lead 160, second group of conductor wires 168 and third group of conductor wires 169 are not coaxial (e.g., nonconcentric, axially offset). Such a configuration is otherwise referred to herein as the second configuration of medical lead 160, and can enable mechanical and/or electrical connection of medical lead 160 to separate, non-coaxial connector portions of medical device 140 (e.g., first connector portion 112 and second connector portion 114 of FIG. 3A, which may be separate, non-coaxial connector portions). In some examples, second group of conductor wires 168 and third group of conductor wires 169 are noncoaxial along the entire length of each of second group of conductor wires 168 and third group of conductor wires 169. In other examples, some or all of second group of conductor wires 168 and third group of conductor wires 169 may be wound around each other (e.g., in a double helix configuration).

In the second configuration, at least a portion of each of second lead body portion 164 and third lead body portion 165 are axially offset (e.g., at least a portion of second lead body portion 164 and third lead body portion 165 do not share a common central longitudinal axis). For example, as shown in FIG. 3C, second lead body portion longitudinal axis 163B and third lead body portion longitudinal axis 163C are not aligned (e.g., are axially offset). In some examples, as shown in FIG. 3C, in the second configuration of medical lead 160, first lead body portion longitudinal axis 163A is coaxial with second lead body portion longitudinal axis 163B, but not coaxial with third lead body portion longitudinal axis 163C.

As shown in the example of FIG. 3A, in the second configuration of medical lead 160, second lead body portion 164 is configured (e.g., sized and/or shaped) to position second group of conductor wires 168 to electrically connect to medical device 140 via first connector portion 112 and third lead body portion 165 is configured (e.g., sized and/or shaped) to position third group of conductor wires 169 to electrically connect to medical device 140 via second connector portion 114. In this way, medical lead 160 is configured to enable electrical connection of conductor wires 166, 168, 169 to medical device 140 having axially offset connector portions (e.g., first connector portion 112 and second connector portion 114 of medical device 140 as illustrated in FIG. 3A).

In some examples, as shown in the example of FIG. 3A and/or FIG. 3C in which medical lead 160 is in the second configuration, first group of electrical contacts 172 of second lead body portion 164 and second group of electrical contacts 173 of third lead body portion 165 can be received by respective, non-coaxial connector portion portions of medical device 140 (e.g., first connector portion 112 and second connector portion 114). In some examples, in the second configuration of medical lead 160, second lead body portion 164 and third lead body portion 165 are positioned such that second lead body portion longitudinal axis 163B and third lead body portion longitudinal axis 163C are substantially parallel (e.g., parallel or nearly parallel to the extent permitted by manufacturing tolerances) but separated by finite gap (e.g., such that second lead body portion longitudinal axis 163B and third lead body portion longitudinal axis 163C cannot be considered the same axis). In this way, second lead body portion 164 and third lead body portion 165 of medical lead 160 can be positioned parallel but offset (e.g., offset in the y-axis direction according to the orthogonal x-y-z axes shown in FIG. 3A and FIG. 3C), which can facilitate mechanical and/or electrical connection to multiple connector portions of medical device 140. In some examples, in the second configuration of medical lead 160, at least one contact of first group of electrical contacts 172 is proximal to at least one contact of second group of electrical contacts 173.

In some examples, some or all of conductor wires of first group of conductor wires 166, second group of conductor wires 168, and/or third group of conductor wires 169 include a material or combination of materials configured to facilitate relatively high flexibility, high axial extensibility, and/or high fatigue resistance. For example, one or more conductor wires of first group of conductor wires 166, second group of conductor wires 168, and/or third group of conductor wires 169 includes a beta-titanium alloy. In some examples, the beta-titanium alloy comprises a Ti-15Mo alloy. Certain beta-titanium alloys, including Ti-15Mo alloy and similar titanium alloys enable a relatively greater number of conductor wires (e.g., twelve conductor wires or greater, including sixteen conductor wires), such as for situations where a relatively high number of individually controlled electrodes are needed in a relatively small space including nerve stimulation and/or sensing from endovascular locations. In some examples, one or more of the conductor wires includes a core material (e.g., a core at a radial center of each conductor wire). The core material can be configured to enhance mechanical robustness. In some examples, the core material includes tantalum.

In some examples, a proximal portion and/or a distal portion of medical lead 160, which may include some or all of conductor wires of first group of conductor wires 166, second group of conductor wires 168, and/or third group of conductor wires 169, includes a beta-titanium alloy (e.g., which may be a second, different beta-titanium alloy than the conductor wires noted above). For example, respective proximal and/or distal portions of one or more of first lead body portion 162, second lead body portion 164 and/or third lead body portion 165 can include the beta-titanium alloy.

In some examples, medical lead 160 includes an electrically insulative material covering at least some portions of medical lead 160. In some examples, one or more of first lead body portion 162, second lead body portion 164 and/or third lead body portion 165 include an electrically insulative material over at least a portion of the coils formed by the conductor wires positioned in each of first lead body portion 162, second lead body portion 164 and/or third lead body portion 165. In some examples, the electrically insulative material includes a tubular-like polymeric covering over one or more portions first lead body portion 162, second lead body portion 164 and/or third lead body portion 165. In some examples, medical lead 160, including one or more portions of first lead body portion 162, second lead body portion 164 and/or third lead body portion 165 includes polyurethane of a suitable durometer (e.g., 55D Shore D hardness). Additionally or alternatively, some or all individual conductor wires of first group of conductor wires 166, second group of conductor wires 168, and/or third group of conductor wires 169 include electrically insulative coatings and/or electrically insulative materials disposed over portions of individual conductor wires. In some examples, some or all individual conductor wires of first group of conductor wires 166, second group of conductor wires 168, and/or third group of conductor wires 169 include Si-polyamide coating.

In some examples, at least a portion of each of first group of conductor wires 166, second group of conductor wires 168, and/or third group of conductor wires 169 include coils that form tubular, wire-like structures. For example, each group of conductor wires may generally form an elongated tube extending along a longitudinal axis and a defining a maximum dimension extending away from the longitudinal axis. In some examples, the coils define a pitch (axial spacing between adjacent individual conductor wires and/or turns of the coil) and/or a pitch angle (e.g., an angle of each individual conductor wires relative to a longitudinal axis of the coil) and/or an. In some examples, an axial spacing between individual adjacent conductor wires of a respective coil is less than a maximum cross-sectional dimension (e.g., diameter) of the individual conductor wires, e.g., such that directly adjacent turns of the wires do not contact each other.

Although the examples of this disclosure include groups of conductor wires as coils, other configurations are possible, such as, but not limited to, bundles, twisted bundles, parallel wire bundles, or other suitable configurations of groups of individual conductor wires. For example, in some examples, and as discussed in relation to at least some examples in this disclosure, at least a portion of first group of conductor wires 166, second group of conductor wires 168, and/or third group of conductor wires 169 can include sections of uncoiled (e.g., straight) conductor wires.

In some examples, one or more of first group of conductor wires 166, second group of conductor wires 168, and/or third group of conductor wires 169 define coils that enable other devices (e.g., guidewires) to be inserted through a lumen of the coils. For example, a coil may include an inner lumen with a diameter large enough to allow a guidewire (or another device) to be inserted into and pass through the lumen. However, in other examples, first group of conductor wires 166, second group of conductor wires 168, and/or third group of conductor wires 169 are configured such that no other devices (e.g., guidewires) can be inserted through and/or within the space occupied by first group of conductor wires 166, second group of conductor wires 168, and/or third group of conductor wires 169. In some instances, a guidewire is not needed to navigate medical lead 160 to a target location within a patient, and the cross-sectional profile of medical lead 160 can be relatively smaller as compared to systems designed to accommodate a guidewire.

Together, first group of conductor wires 166, second group of conductor wires 168, and/or third group of conductor wires 169 facilitate electrical connection of electrodes 170 to medical device 140. In some examples, each of electrodes 170 is configured to be individually (e.g., separately, independently) controlled via medical device 140 to deliver electrical stimulation therapy and/or sense a patient parameter. In the example of FIG. 3A, each electrode of electrodes 170 is electrically connected to at least one conductor wire of first group of conductor wires 166. Because of the relatively high number of first group of conductor wires 166, a relatively high number of corresponding electrodes may be connected to and used in conjunction with medical device 140, while still maintaining a relatively low endovascular device profile (e.g., relative to two separate, noncoaxial groups of conductor wires) that facilitates positioning of electrodes 170 in vasculature of a patient.

In some examples, electrodes 170 include at least twelve of electrodes 170 (e.g., sixteen of electrodes 170), although other suitable numbers of electrodes are provided in other examples (e.g., four, six, eight, twelve, fourteen, sixteen, eighteen, twenty, or more electrodes). In some examples, a number of electrodes of electrodes 170 equals a number of conductor wires of first group of conductor wires 166 (e.g., sixteen electrodes for sixteen conductor wires). However, in other examples, more than one electrode of electrodes 170 is connected to each conductor wire, such that a number of electrodes is greater than a number of conductor wires of first group of conductor wires 166 (e.g., in examples where at least some electrodes are shorted or ganged together). In some examples, the number of second group of conductor wires 168 and the number of third group of conductor wires 169 equals the number of first group of conductor wires 166. In some examples, each of second group of conductor wires 168 and third group of conductor wires 169 each include at least six conductor wires (e.g., eight conductor wires).

In some examples, as shown in the examples of FIG. 3B and FIG. 3C, medical lead 160 includes a connection body 152 proximate junction 153 (e.g., at the interface of at least first lead body portion 162 and second lead body portion 164). In some examples, connection body 152 is formed via molding (e.g., injection molding), such that connection body 152 can be considered a molded body. In some examples, connection body 152 is configured to enable third lead body portion 165 to be positioned entirely proximally of second lead body portion 164 such that second lead body portion 164 and third lead body portion 165 can be positioned in sequence (e.g., in sequence along the x-axis direction according to the orthogonal x-y-z axes in FIG. 3B) and coaxial. For example, at least a portion of connection body 152 (e.g., a connection body proximal end 155A as shown in FIG. 3D) can be coupled to a portion of third lead body portion 165 to enable third lead body portion 165, including all of second group of electrical contacts 173, to be positioned proximally of a proximal-most end of second lead body portion 164. In this way, connection body 152 can enable medical lead 160 to transform to a configuration in which at least second lead body portion 164 and third lead body portion 165 are coaxial, which may facilitate easier movement of medical lead 160 relative to and/or within a lumen of a delivery catheter and/or sheath, as discussed throughout this disclosure.

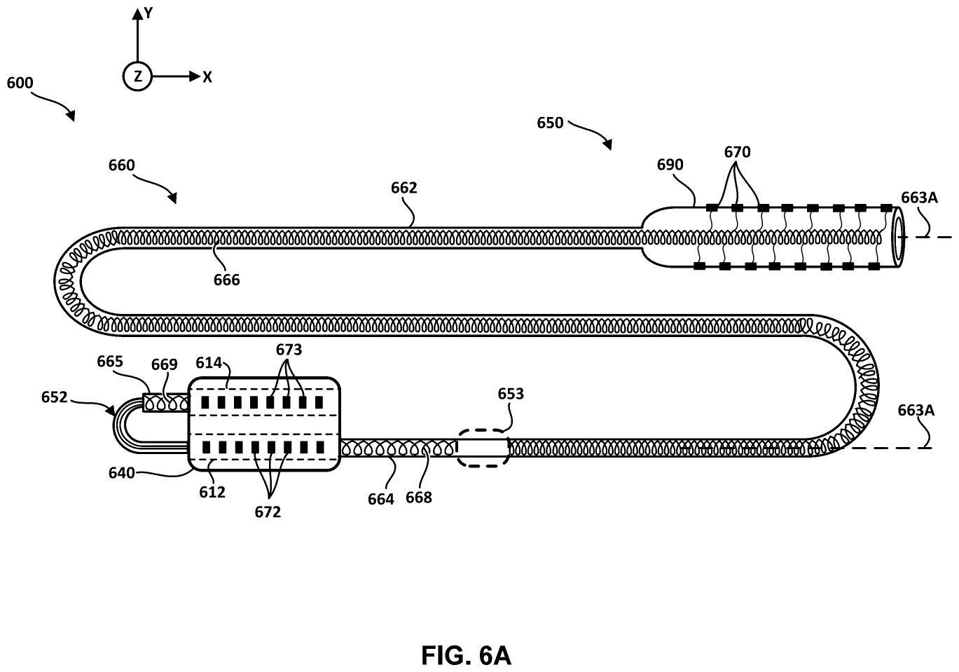

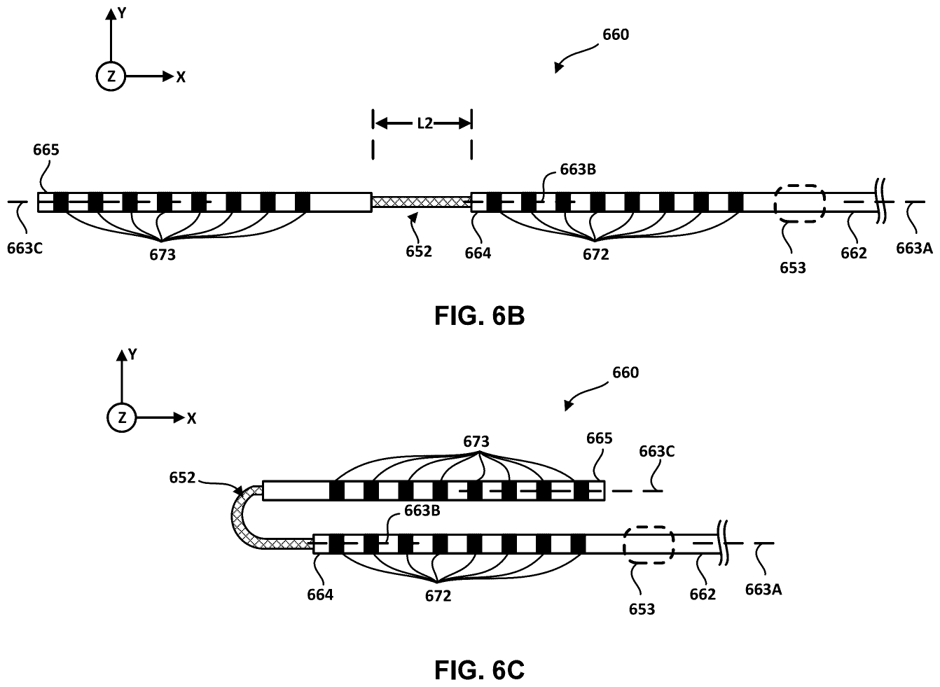

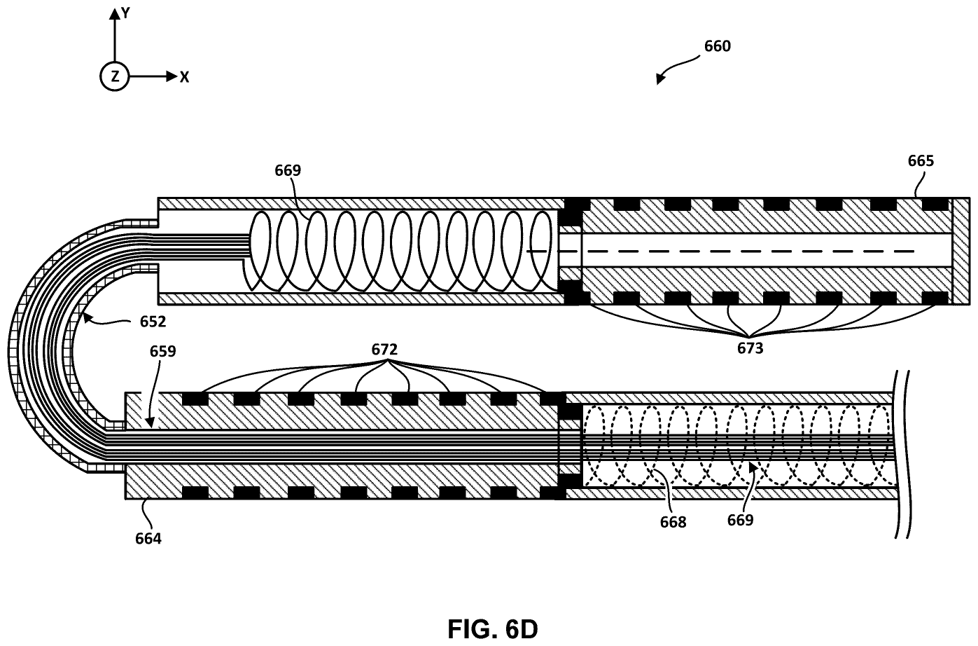

In some examples, as shown in the example of FIG. 3B, connection body 152 is shaped, sized, and/or otherwise configured to enable a distal end of third lead body portion 165 to be positioned proximal of a proximal end of second lead body portion 164 by at least a distance L1 (e.g., wherein L1 is a finite distance measured along the x-axis direction according to orthogonal x-y-z axes in FIG. 3B). In other words, in some examples, in the first configuration of medical lead 160, second lead body portion 164 and third lead body portion 165 can be separated by a gap of distance L1. In some examples, distance L1 is at least 1 mm. In some examples, distance L1 is 1 mm to 3 mm. Such a gap can ensure that second lead body portion 164 and third lead body portion 165 can be positioned in sequence in a linear configuration. In some examples, connection body 152 is sufficiently long and/or distance L1 is sufficient to enable third lead body portion 165 to be received in a connector portion of a medical device in which the opening of the connector portion is on an opposite side of the medical device as compared the opening of the connector portion that receives second lead body portion 164 (e.g., like medical device 660 shown and described in relation to FIG. 6A).

In some examples, at least a portion of connection body 152 is positioned at junction 153. In some examples, as shown in FIG. 3B and FIG. 3C, junction 153 is at the interface of where first lead body portion 162 is mechanically coupled to second lead body portion 164 and third lead body portion 165 (e.g., such that junction 153 is at the interface between first lead body portion 162 and each of second lead body portion 164 and third lead body portion 165).

FIG. 3D illustrates an example connection body 152. In the example of FIG. 3D, connection body 152 extends from connection body proximal end 155A and a connection body distal end 155B and includes a first connection body portion 154 and a second connection body portion 156. In some examples, with reference to FIG. 3B and FIG. 3C, first connection body portion 154 is configured to mechanically couple to the lead body of medical lead 160 (e.g., at junction 153 that separates first lead body portion 162 from second lead body portion 164 and third lead body portion 165). In some examples, with reference to FIG. 3B and FIG. 3C, second connection body portion 156 is configured to extend parallel to and along at least a portion of the lead body of medical lead 160 (e.g., in the example of FIG. 3B, second connection body portion 156 extends along an outer surface of second lead body portion 164). In some examples, at least second connection body portion 156 defines a length sufficient to position third lead body portion 165 entirely proximal of second lead body portion 164.

In some examples, at least a portion of connection body 152 is configured to receive at least some of the plurality of conductor wires 166, 168, 169. In some examples, at least third group of conductor wires 169 are positioned along and/or within second connection body portion 156. In some examples, second connection body portion 156 defines a lumen 157 configured to receive and/or house at least third group of conductor wires 169, e.g., to separate conductor wires 169 from an external environment, such as blood flow in a blood vessel of a patient. In some examples, connection body 152 includes an electrically insulative material configured to electrically insulate at least third group of conductor wires 169 from the environment outside of (e.g., radially outside of) connection body 152.

In some examples, at least a portion of connection body 152 is configured to reversibly deform to accommodate transformation of medical lead 160 between the first configuration and the second configuration (e.g., the first configuration of medical lead 160 shown and described in connection with FIG. 3B and the second configuration of medical lead 160 as shown and described connection with FIG. 3A and/or FIG. 3C). For example, as illustrated between FIG. 3B and FIG. 3C, at least a portion of connection body 152 (e.g., second connection body portion 156 as shown in FIG. 3D) can be configured to flex, bend, or otherwise deform to accommodate transformation of medical lead 160 between the first configuration of medical lead 160 in which second lead body portion 164 and third lead body portion 165 are coaxial and the second configuration of medical lead 160 in which second lead body portion 164 and third lead body portion 165 are not coaxial (e.g., axially offset).

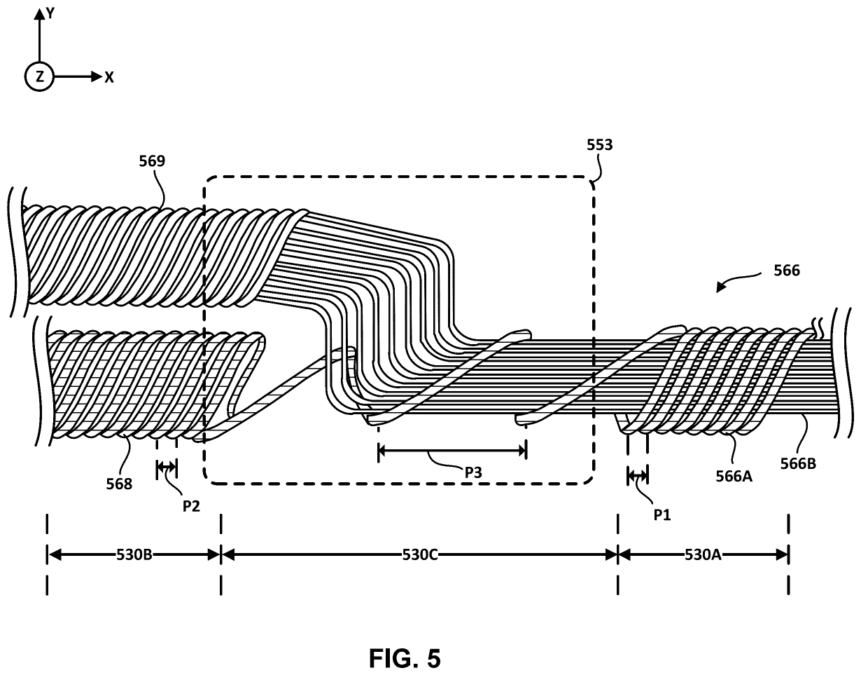

FIG. 4 and FIG. 5 illustrate example configurations of conductor wires at a junction (e.g., such as junction 153 of FIG. 3A, FIG. 3B, and FIG. 3C) that enable at least a first group of conductor wires to split to form at least respective non-coaxial second and third groups of conductor wires. As discussed previously, although the term “split” is used throughout this disclosure to describe the bifurcation or other separation of wires into different respective groups, conductor wires forming different groups of conductor wires can be continuous wires. In general, the examples of FIG. 4 and FIG. 5 illustrate configurations of coils that enable bifurcation of a single group of conductor wires into two different groups of conductor wires. However, it is understood that other configurations of conductor wires that enable splitting of a single group of conductor wires into two different groups of conductor wires are contemplated.

In the example of FIG. 4, a first group of conductor wires 466 includes a first subset 466A and a second subset 466B of first group of conductor wires 466. First group of conductor wires 466 splits to form a second group of conductor wires 468 and a third group of conductor wires 469 at a junction 453. As shown in the example of FIG. 4, second group of conductor wires 468 and third group of conductor wires 469 are not coaxial and nonconcentric. First group of conductor wires 466, second group of conductor wires 468, third group of conductor wires 469, and junction 453 are examples of first group of conductor wires 166, second group of conductor wires 168, third group of conductor wires 169, and junction 153 of FIG. 3A, respectively, and may be configured similarly except as described herein.

As shown in the example of FIG. 4, the respective positions of each of first group of conductor wires 466, second group of conductor wires 468, and third group of conductor wires 469 can also be described with respect to a first section 430A (e.g., in which first section 430A includes first group of conductor wires 466), a second section 430B (e.g., in which second section 430B includes second group of conductor wires 468 and third group of conductor wires 469), and a third section 430C between first section 430A and second section 430B (e.g., in which third section 430C includes the transition between first group of conductor wires 466 and each of second group of conductor wires 468 and third group of conductor wires 469). Sections 430A, 430B, 430C can be, for example, longitudinal sections of conductor wires of a medical lead.

In the example of FIG. 4, at least first subset 466A of first group of conductor wires 466 form a first coil. In some examples, second subset 466B of first group of conductor wires 466 are positioned within the first coil (e.g., as shown at first section 430A). In some examples, second subset 466B of first group of conductor wires 466 positioned within the first coil are uncoiled (e.g., straight or substantially straight, to the extent permitted by manufacturing tolerances). Such configurations in which first group of conductor wires 466 includes an outer coil formed from first subset 466A with an uncoiled second subset 466B positioned and extending within the coil can exhibit a relatively a smaller cross-sectional profile as compared to other configurations (e.g., a single coil of wires and/or dual coils).

In some examples, second group of conductor wires 468 forms a second coil, and each of second group of conductor wires 468 is an extension of a respective one of conductor wires from first subset 466A of the first group of conductor wires 466. In some examples, third group of conductor wires 469 forms a third coil, and each of third group of conductor wires 469 is an extension of a respective one of conductor wires from second subset 466B of the first group of conductor wires 466. The second coil and the third coil formed by each of second group of conductor wires 468 and third group of conductor wires 469 are positioned generally at second section 430B in the example of FIG. 4. The respective, non-coaxial coils formed by each of second group of conductor wires 468 and third group of conductor wires 469 facilitates electrical connection to respective non-coaxial connector portions of a medical device (e.g., first connector portion 112 and second connector portion 114 of medical device 140 of the example of FIG. 3A).

In some examples, as shown in the example of FIG. 4, a section of uncoiled conductor wires (e.g., shown generally at third section 430C between first section 430A and second section 430B) separates the first coil (e.g., the first coil formed by first subset 466A of first group of conductor wires 466) and the second coil (e.g., the second coil formed by second group of conductor wires 468) to enable the second subset 466B of the first group of conductor wires 466 to exit (e.g., such as to be positioned radially outside of) the first coil. The section of uncoiled conductor wires (e.g., generally shown at third section 430C) can be sufficiently long to enable all of second subset 466B of the first group of conductor wires 466 to exit the first coil formed by first subset 466A of first group of conductor wires 466.

Such configurations of conductor wires 466, 468, and 469 as shown and described with respect to FIG. 4 can enable use of a continuous (e.g., unbroken) length of wires while having a single distal lead body that splits to form separate, non-coaxial lead body portions that can connect to separate, axially offset connector portions of a medical device (e.g., first connector portion 112 and second connector portion 114 as shown and described with respect to FIG. 3A).

In the example of FIG. 5, a first group of conductor wires 566 includes a first subset 566A and a second subset 566B of first group of conductor wires 566. First group of conductor wires 566 splits to form a second group of conductor wires 568 and a third group of conductor wires 569 at a junction 553. As shown in the example of FIG. 5, second group of conductor wires 568 and third group of conductor wires 569 are not coaxial and nonconcentric. First group of conductor wires 566, second group of conductor wires 568, third group of conductor wires 569, and junction 553 may be examples of first group of conductor wires 166, second group of conductor wires 168, third group of conductor wires 169, and junction 153 of FIG. 3A, respectively, and may be configured similarly except as described herein.

As shown in the example of FIG. 5, the respective positions of each of first group of conductor wires 566, second group of conductor wires 568, and third group of conductor wires 569 can also be described with respect to a first section 530A (e.g., in which first section 530A includes first group of conductor wires 566), a second section 530B (e.g., in which second section 530B includes second group of conductor wires 568 and third group of conductor wires 569), and a third section 530C between first section 530A and second section 530B (e.g., in which third section 530C includes the transition between first group of conductor wires 566 and each of second group of conductor wires 568 and third group of conductor wires 569). Sections 530A, 530B, 530C can be, for example, longitudinal sections of conductor wires of a medical lead.

In the example of FIG. 5, at least first subset 566A of first group of conductor wires 566 form a first coil defining a first pitch P1 (e.g., wherein pitch P1 represents a distance between directly adjacent turns of the first coil formed by at least first subset 566A of first group of conductor wires 566). In some examples, second subset 566B of first group of conductor wires 566 are positioned within the first coil (e.g., as shown at first section 530A). In some examples, second subset 566B of first group of conductor wires 566 positioned within the first coil are uncoiled (e.g., straight or substantially straight, to the extent permitted by manufacturing tolerances). Such configurations in which first group of conductor wires 566 includes an outer coil formed from first subset 566A with uncoiled second subset 566B positioned and extending within the coil can exhibit a relatively smaller cross-sectional profile as compared to other configurations (e.g., as compared a single coil of all wires, or a dual-coil configuration).

In some examples, second group of conductor wires 568 forms a second coil defining a second pitch P2 (e.g., wherein pitch P2 represents a distance between directly adjacent turns of the second coil formed by second group of conductor wires 568). In the example of FIG. 5, each conductor wire of second group of conductor wires 568 is an extension of a respective one of conductor wires from first subset 566A of the first group of conductor wires 566. In some examples, third group of conductor wires 569 forms a third coil, and each conductor wire of third group of conductor wires 569 is an extension of a respective one of conductor wires from second subset 566B of the first group of conductor wires 566. The second coil and the third coil formed by each of second group of conductor wires 568 and third group of conductor wires 569 are positioned generally at second section 530B in the example of FIG. 5. The respective, non-coaxial coils formed by each of second group of conductor wires 568 and third group of conductor wires 569 facilitates electrical connection to respective non-coaxial connector portions of a medical device (e.g., first feed through 112 and second connector portion 114 of medical device 140 of the example of FIG. 3A).

In some examples, a section of wider-pitch coiled conductor wires (e.g., shown generally at third section 530C, which is axially between first section 530A and second section 530B) separates the first coil (e.g., the first coil formed by first subset 566A of first group of conductor wires 566) and the second coil (e.g., the second coil formed by second group of conductor wires 568). Such a section of wider-pitch coiled conductor wires can enable the second subset 566B of the first group of conductor wires 566 to exit (e.g., such as to be positioned radially outside of) the first coil. The section of wider-pitch coiled conductor wires (e.g., generally shown at third section 530C) defines a pitch P3, which can be sufficiently large enough to enable all of second subset 566B of the first group of conductor wires 566 to exit the first coil formed by first subset 566A of first group of conductor wires 566. In some examples, pitch P3 of the section of wider-pitch coiled conductor wires is greater than first pitch P1 and second pitch P2 such as to enable second subset 566B of the first group of conductor wires 566 to exit the first coil formed by first subset 566A of first group of conductor wires 566.

Such configurations of conductor wires 566, 568, and 569 as shown and described with respect to FIG. 5 can enable use of a continuous (e.g., unbroken) length of wires while having a single distal lead body that splits to form separate, non-coaxial lead body portions that can connect to separate, axially offset connector portions of a medical device (e.g., first connector portion 112 and second connector portion 114 as shown and described with respect to FIG. 3A).

FIG. 6A, FIG. 6B, FIG. 6C, and FIG. 6D illustrate an example endovascular therapy system 600 and various components thereof. Endovascular therapy system 600 is an example of therapy system 10 of FIG. 1. Endovascular therapy system 600 includes a medical device 640, a medical lead 660, and a plurality of electrodes 670 (which are examples of medical device 14, endovascular device 16, and electrodes 17, respectively, as shown and described in connection with FIG. 1, respectively).

In the example of FIG. 6A, medical lead 660 includes a first lead body portion 662, a second lead body portion 664, and a third lead body portion 665 (e.g., where first lead body portion 662, second lead body portion 664, and third lead body portion 665 collectively form a lead body of medical lead 660). In some examples, first lead body portion 662 is a distal portion (e.g., a distal-most portion) of medical lead 660. In some examples, second lead body portion 664 and third lead body portion 665 together form a proximal portion of medical lead 660. In some examples, as shown in the examples of FIG. 6A, FIG. 6B, and FIG. 6C, second lead body portion 664 is an integral extension of first lead body portion 662 (e.g., such that first lead body portion 662 transitions to form second lead body portion 664 at a junction 653). In other examples, first lead body portion 662 is formed separately from and mechanically coupled to second lead body portion 664 at junction 653 (e.g., where junction 653 is at the interface of first lead body portion 662 and second lead body portion 664).

As shown in the example of FIG. 6A, endovascular therapy system 600 includes a plurality of electrodes 670 and an expandable structure 690 at a distal portion 650 of medical lead 660. In some examples, plurality of electrodes 670 and an expandable structure 690 is configured similar to electrodes 170 and expandable structure 190 of FIG. 3A.

As shown in the example of FIG. 6A, as well as FIG. 6B and FIG. 6C, first lead body portion 662 portion defines a first lead body portion longitudinal axis 663A, which may be a central longitudinal axis extending through a radial center of first lead body portion 662. As shown in the examples of FIG. 6B and FIG. 6C, second lead body portion 664 defines a second lead body portion longitudinal axis 663B, which may be a central longitudinal axis extending through a radial center of second lead body portion 664, and the third lead body portion 665 defines a third lead body portion longitudinal axis 663C, which may be a central longitudinal axis extending through a radial center of third lead body portion 665. Each of first lead body portion longitudinal axis 663A, second lead body portion longitudinal axis 663B, and third lead body portion longitudinal axis 663C can be aligned (e.g., axially aligned, or coaxial), or offset (e.g., axially offset, which can include parallel) depending on the configuration of medical lead 660.

Medical lead 660 includes a plurality of conductor wires 666, 668, 669 (shown individually as a first group of conductor wires 666, a second group of conductor wires 668, and a third group of conductor wires 669, but collectively referred to herein as plurality of conductor wires 666, 668, 669). In the example of FIG. 6A, first lead body portion 662 includes (e.g., is configured to receive and/or otherwise incorporates) first group of conductor wires 666, second lead body portion 664 includes (e.g., is configured to receive and/or otherwise incorporates) second group of conductor wires 668, and third lead body portion 665 includes (e.g., is configured to receive and/or otherwise incorporates) third group of conductor wires 669.

As shown in the example of FIG. 6A, each of electrodes 670 is electrically connected to at least one conductor wire of first group of conductor wires 666. In some examples, as illustrated in the FIG. 6A, first group of conductor wires 666 transitions to form second group of conductor wires 668 and third group of conductor wires 669 at junction 653. Such transitioning can facilitate connection of second group of conductor wires 668 and third group of conductor wires 669 to separate connector portions of medical device 640. As illustrated in FIG. 6D, third group of conductor wires 669 can be configured to extend through second lead body portion 664 to third lead body portion 665. In some examples, as discussed in relation to the example of FIG. 6D, second lead body portion 664 defines a lumen configured to receive group of conductor wires 669 therethrough.

Although the term “transition” is used herein to describe a termination of first group of conductor wires 666 and a beginning of each of second group of conductor wires 668 and third group of conductor wires 669, second group of conductor wires 668 and third group of conductor wires 669 can be continuous extensions of the respective subsets of wires that form first group of conductor wires 666. For example, each wire of second group of conductor wires 668 can be a continuous extension of one wire of a first subset of first group of conductor wires 666 and each wire of third group of conductor wires 669 can be a continuous extension of one wire of a second subset of first group of conductor wires 666. In some examples, each conductor wire of the plurality of conductor wires 666, 668, 669 extends continuously between electrodes 670 and medical device 640 (e.g., such as to electrically connect each of electrodes 670 to circuitry of medical device 640).

In some examples, the transition of first group of conductor wires 666 to each of second group of conductor wires 668 and third group of conductor wires 669 at junction 653 includes a change in shape and/or orientation of the individual conductor wires. For example, first group of conductor wires 666 can include a single coil or dual concentric coils of conductor wires that transitions (e.g., at junction 653) to form a single coil with some straight conductor wires within the coil (e.g., wherein second group of conductor wires 668 includes the single coil and third group of conductor wires 669 includes the straight conductor wires within the coil formed by second group of conductor wires 668). The uncoiled within coiled configuration of third group of conductor wires 669 and second group of conductor wires 668 can facilitate mechanical robustness (e.g., fatigue resistance) of the conductor wires while also maintaining a relatively low cross-sectional profile of the group of conductor wires.

In some examples, at least a portion of second lead body portion 664 and/or third lead body portion 665 are configured to be positioned outside of the vasculature to facilitate electrical connection of the plurality of conductor wires 666, 668, 669 to medical device 640. Medical device 640 may be placed in an extravascular portion of the chest or external to patient 12. In some examples, such as in instances of temporary electrical stimulation therapy, sensing, and/or screening, medical device 640 is configured to be positioned externally of patient 12.

In some examples, medical device 640 is configured to receive one or more portions of medical lead 660, such as for electrically connecting circuitry of medical device 640 (e.g., processing circuitry, therapy generation circuitry, and/or sensing circuitry) to plurality of conductor wires 666, 668, 669. As shown in the example of FIG. 6A, medical device 640 includes a first connector portion 612 and a second connector portion 614. However, medical device 640 can include any suitable number of connector portions (e.g., one connector portion, two connector portions, three connector portions, four connector portions, or more). In some examples, first connector portion 612 is configured to receive a first portion of medical lead 660 (e.g., second lead body portion 664 of medical lead 660) and second connector portion 614 is configured to receive a second portion of medical lead 660 (e.g., third lead body portion 665 of medical lead 660). In some examples, one or more of first connector portion 612 and second connector portion 614 can extend entirely through medical device 640 (e.g., as through-holes), such that portions of medical lead 660 can be inserted into one or more of first connector portion 612 or second connector portion 614 from at least two sides of medical device 640 and/or such that at least a portion of medical lead 660 can extend entirely through medical device 640.

In the example of FIG. 6A, second group of conductor wires 668 is configured to electrically connect to medical device 640 via first connector portion 612, and third group of conductor wires 669 is configured to electrically connect to medical device 640 via second connector portion 614. In the example of FIG. 6A, second lead body portion 664 of medical lead 160 includes a first group of electrical contacts 672 that are electrically connected to second group of conductor wires 668 and facilitate electrical connection of at least some electrodes 670 to medical device 640 via first connector portion 612. In some examples, each of first group of electrical contacts 672 is electrically connected to a respective one of second group of conductor wires 668. As shown in the example of FIG. 6B and FIG. 6C, first group of electrical contacts 672 are spaced apart along second lead body portion longitudinal axis 663B. In some examples, first group of electrical contacts 672 are disposed on a proximal portion (e.g., a proximal-most portion) of second lead body portion 664.

In some examples, medical lead 660 includes a second group of electrical contacts 673 that are electrically connected to third group of conductor wires 669 and facilitate electrical connection of at least some electrodes 670 to medical device 640 via second connector portion 614. In some examples, each of second group of electrical contacts 673 is electrically connected to a respective one of third group of conductor wires 669. As shown in the example of FIG. 6B and FIG. 6C, second group of electrical contacts 673 are spaced apart along third lead body portion longitudinal axis 663C. In some examples, second group of electrical contacts 673 are disposed on a proximal portion (e.g., a proximal-most portion) of third lead body portion 665.

In some examples, first group of electrical contacts 672 and second group of electrical contacts 673 are configured to engage respective features (e.g., corresponding electrical contacts) of first connector portion 612 and second connector portion 614, such as for electrically connecting second group of conductor wires 668 and third group of conductor wires 669 to circuitry (e.g., processing circuitry, therapy generation circuitry, sensing circuitry, as discussed in relation to FIG. 2) of medical device 640. For example, in some examples, second lead body portion 664 is configured to be received in first connector portion 612 to electrically connect second group of conductor wires 668 to a first set of electrical contacts of medical device 640 (not shown in FIG. 6A). In some examples, third lead body portion 665 is configured to be received in second connector portion 614 of the medical device 640 to electrically connect third group of conductor wires 669 to a second set of electrical contacts of medical device 640 (not shown in FIG. 6A). In this way, second group of conductor wires 668 is configured to electrically connect to medical device 640 via first group of electrical contacts 672 and third group of conductor wires 669 is configured to electrically connect to medical device 640 via second group of electrical contacts 673.

In some examples, second lead body portion 664 and/or third lead body portion 665 are configured to mechanically couple to medical device 640. In some examples, medical device 640 is configured to receive portions of second lead body portion 664 and/or third lead body portion 665 and/or retain portions of second lead body portion 664 and/or third lead body portion 665 within medical device 640. For example, medical device 640 can include a header (e.g., similar to header 11 in the example of FIG. 1) configured to receive and/or retain portions of second lead body portion 664 and/or third lead body portion 665. In some examples, second lead body portion 664, third lead body portion 665 and/or medical device 640 include features (e.g., mating features) to facilitate mechanical coupling (e.g., snap fit or interference features). In some examples, medical device 640 includes one or set screws for mechanically coupling second lead body portion 664 and/or third lead body portion 665 to medical device 640. Second lead body portion 664, third lead body portion 665 and/or medical device 640 can include any suitable features for mechanical connection, including Bal Seal® connectors, spring connectors, push fittings, or other suitable connectors.

As illustrated in the examples of FIG. 6A, FIG. 6B, and FIG. 6C, medical lead 660 is configured to transform between at least a first configuration and second configuration. In the first configuration of medical lead 660 as illustrated in FIG. 6B, medical lead 660 is configured to slide and/or move relative to (e.g., within) a delivery catheter and/or sheath (e.g., in which a delivery catheter and/or sheath radially surrounds medical lead 660 and is retracted overtop of medical lead 660), at least because portions of medical lead 660 are arranged linearly and/or coaxially. In some examples, in the first configuration of medical lead 660, a substantial portion of medical lead 660 (e.g., including at least second lead body portion 664, third lead body portion 665, and a majority of first lead body portion 662) defines a substantially uniform (e.g., uniform or nearly uniform to the extent permitted by manufacturing tolerances) outer cross-sectional dimension (e.g., an outer diameter), which can facilitate relatively easier movement of medical lead 660 within a delivery catheter and/or sheath.

In the second configuration of medical lead 660, as illustrated in at least FIG. 6A and FIG. 6C, portions of medical lead 660 (e.g., second lead body portion 664 and third lead body portion 665) are configured to mechanically and/or electrically couple to respective, non-coaxial connector portions (e.g., first connector portion 612 and second connector portion 614) of medical device 640. This ability of medical lead 660 to transform between such first configuration and second configuration can enable medical lead 660 to move (e.g., slide) more easily relative to and/or within a lumen a delivery catheter and/or sheath (e.g., as compared to other medical leads that do not have a configuration in which all or a majority of portions of the medical lead are able to be linearly and/or coaxially arranged), while also enabling medical lead 660 to electrically and/or mechanically couple with multi-connector portion medical devices (e.g., such as medical device 640) configured to receive respective, non-coaxial portions. Additionally, such configurations of medical lead 660 can advantageously reduce or even eliminate the need for supplemental lead connectors and/or other lead extensions that would otherwise be needed for medical lead 660 to mechanically and/or electrically connect to medical device 640.

The example of FIG. 6B illustrates a portion of medical lead 660 in which medical lead 660 is in the first configuration. In the first configuration, second lead body portion 664 and third lead body portion 665 are coaxial (e.g., second lead body portion 664 and third lead body portion 665 share a common central longitudinal axis) or at least substantially coaxial. For example, as shown in FIG. 6B, second lead body portion longitudinal axis 663B and third lead body portion longitudinal axis 663C are coaxial (e.g., aligned such that they can be considered a single axis). In some examples, as shown in FIG. 6B, in the first configuration, second lead body portion longitudinal axis 663B and third lead body portion longitudinal axis 663C are also coaxial with first lead body portion longitudinal axis 663A (e.g., such that all lead body portions of medical lead 660 share a common central longitudinal axis).

In some examples, when medical lead 660 is in the first configuration, at least second lead body portion 664 and third lead body portion 665 are configured to pass through a lumen of the delivery catheter. In such examples, in the first configuration, second lead body portion 664 and third lead body portion 665 are configured to enable a delivery catheter (not shown in FIG. 6B) to be retracted over each second lead body portion 664 and third lead body portion 665 in sequence. Such ability of medical lead 660 to transform to the first configuration illustrated in FIG. 6B in which at least second lead body portion 664 and third lead body portion 665 are coaxial and/or are positioned in linear succussion can facilitate relatively easier movement of medical lead 660 relative to and/or within a lumen of the delivery catheter and/or sheath (e.g., because the delivery catheter and/or sheath can be more easily retracted from overtop the endovascular device), and/or enable use of a relatively smaller delivery catheter and/or sheath (e.g., having a smaller cross-sectional dimension, such as a smaller diameter).

In some examples, as shown in the example of FIG. 6B in which medical lead 660 is in the first configuration, all of second group of electrical contacts 673 of third lead body portion 665 are proximal to all electrical contacts of first group of electrical contacts 672 of second lead body portion 664. In this way, second lead body portion 664 and third lead body portion 665 of medical lead 660 can be positioned in sequence (e.g., in sequence along the x-axis direction according to the orthogonal x-y-z axes shown in FIG. 6B), which can facilitate relatively easier movement of medical lead 660 relative to a delivery catheter and/or sheath.

The examples of FIG. 6A and FIG. 6C illustrate a portion of medical lead 660 in which medical lead 660 is in the second configuration. As shown in the example of FIG. 6A, in the second configuration of medical lead 660, at least a portion of second group of conductor wires 668 and third group of conductor wires 669 are not coaxial (e.g., nonconcentric, axially offset). Such a configuration is otherwise referred to herein as the second configuration of medical lead 660, and can enable mechanical and/or electrical connection of medical lead 660 to separate, non-coaxial connector portions of medical device 640 (e.g., first connector portion 612 and second connector portion 614 as shown in FIG. 6A, which may be separate, non-coaxial connector portions).

In the second configuration, at least a portion of each of second lead body portion 664 and third lead body portion 665 are axially offset (e.g., at least a portion of each of second lead body portion 664 and third lead body portion 665 do not share a common central longitudinal axis). For example, as shown in FIG. 6C, second lead body portion longitudinal axis 663B and third lead body portion longitudinal axis 663C are not aligned (e.g., are axially offset). In some examples, as shown in FIG. 6C, in the second configuration of medical lead 660, first lead body portion longitudinal axis 663A is coaxial with second lead body portion longitudinal axis 663B, but not coaxial with third lead body portion longitudinal axis 663C.

As shown in the example of FIG. 6A, in the second configuration of medical lead 660, second lead body portion 664 is configured (e.g., sized and/or shaped) to position second group of conductor wires 668 to electrically connect to medical device 640 via first connector portion 612 and third lead body portion 665 is configured (e.g., sized and/or shaped) to position third group of conductor wires 669 to electrically connect to medical device 640 via second connector portion 614. In this way, medical lead 660 is configured to enable electrical connection of conductor wires 666, 668, 669 to medical device 640 having axially offset connector portions (e.g., first connector portion 612 and second connector portion 614 of medical device 640 as illustrated in FIG. 6A).

In some examples, as shown in the example of FIG. 6A and/or FIG. 6C in which medical lead 660 is in the second configuration, first group of electrical contacts 672 of second lead body portion 664 and second group of electrical contacts 673 of third lead body portion 665 can be received by respective, non-coaxial connector portion portions of medical device 640 (e.g., first connector portion 612 and second connector portion 614). In some examples, in the second configuration of medical lead 660, second lead body portion 664 and third lead body portion 665 are positioned such that second lead body portion longitudinal axis 663B and third lead body portion longitudinal axis 663C are substantially parallel (e.g., parallel or nearly parallel to the extent permitted by manufacturing tolerances) but separated by finite gap (e.g., such that second lead body portion longitudinal axis 663B and third lead body portion longitudinal axis 663C cannot be considered the same axis). In this way, second lead body portion 664 and third lead body portion 665 of medical lead 660 can be positioned parallel but offset (e.g., offset in the y-axis direction according to the orthogonal x-y-z axes shown in FIG. 6A and FIG. 6C), which can facilitate mechanical and/or electrical connection to multiple connector portions of medical device 640. In some examples, in the second configuration of medical lead 660, at least one contact of first group of electrical contacts 672 is proximal to at least one contact of second group of electrical contacts 673.

Conductor wires of first group of conductor wires 666, second group of conductor wires 668, and/or third group of conductor wires 669 can include a material, combination of materials, or configuration of materials like plurality of conductor wires 166, 168, 169 as described in connection with FIG. 3A, FIG. 3B, and FIG. 3C.

In some examples, medical lead 660 includes an electrically insulative material covering at least some portions of medical lead 660. In some examples, one or more of first lead body portion 662, second lead body portion 664 and/or third lead body portion 665 include an electrically insulative material over at least a portion of the coils formed by the conductor wires of each of first lead body portion 662, second lead body portion 664 and/or third lead body portion 665. In some examples, the electrically insulative material includes a tubular-like polymeric covering over one or more portions first lead body portion 662, second lead body portion 664 and/or third lead body portion 665. In some examples, medical lead 660, including one or more portions of first lead body portion 662, second lead body portion 664 and/or third lead body portion 665 includes polyurethane of a suitable durometer (e.g., 55D Shore D hardness). Additionally or alternatively, some or all individual conductor wires of first group of conductor wires 666, second group of conductor wires 668, and/or third group of conductor wires 669 include electrically insulative coatings and/or electrically insulative materials disposed over portions of individual conductor wires. In some examples, some or all individual conductor wires of first group of conductor wires 666, second group of conductor wires 668, and/or third group of conductor wires 669 include Si-polyamide coating.

In some examples, at least a portion of each of first group of conductor wires 666, second group of conductor wires 668, and/or third group of conductor wires 669 include coils that form tubular, wire-like structures. For example, each group of conductor wires may generally form an elongated tube extending along a longitudinal axis and a defining a maximum dimension extending away from the longitudinal axis. In some examples, the coils define a pitch (axial spacing between adjacent individual conductor wires and/or turns of the coil) and/or a pitch angle (e.g., an angle of each individual conductor wires relative to a longitudinal axis of the coil) and/or an. In some examples, an axial spacing between individual adjacent conductor wires of a respective coil is less than a maximum cross-sectional dimension (e.g., diameter) of the individual conductor wires, e.g., such that directly adjacent turns of the wires do not contact each other.

Although the examples of this disclosure include groups of conductor wires as coils, other configurations are possible, such as, but not limited to, uncoiled bundles of wires, twisted bundles, parallel wire bundles, or other suitable configurations of groups of individual conductor wires. For example, as discussed in relation to FIG. 6D, at least a portion of third group of conductor wires 669 can include an uncoiled bundle of conductor wires. In some examples, a portion of third group of conductor wires 669 include substantially straight or straight conductor wires.

In some examples, one or more of first group of conductor wires 666, second group of conductor wires 668, and/or third group of conductor wires 669 define coils that enable other devices (e.g., guidewires) to be inserted through a lumen of the coils. For example, a coil may include an inner lumen with a diameter large enough to allow a guidewire (or another device) to be inserted into and pass through the lumen. However, in other examples, first group of conductor wires 666, second group of conductor wires 668, and/or third group of conductor wires 669 are configured such that no other devices (e.g., guidewires) can be inserted through and/or within the space occupied by first group of conductor wires 666, second group of conductor wires 668, and/or third group of conductor wires 669. In some instances, a guidewire is not needed to navigate medical lead 660 to a target location within a patient, and the cross-sectional profile of medical lead 660 can be relatively smaller as compared to systems designed to accommodate a guidewire.

Together, first group of conductor wires 666, second group of conductor wires 668, and/or third group of conductor wires 669 facilitate electrical connection of electrodes 670 to medical device 640. In some examples, each of electrodes 670 is configured to be individually (e.g., separately, independently) controlled via medical device 640 to deliver electrical stimulation therapy and/or sense a patient parameter. In the example of FIG. 6A, each electrode of electrodes 670 is electrically connected to at least one conductor wire of first group of conductor wires 666. Because of the relatively high number of first group of conductor wires 666, a relatively high number of corresponding electrodes may be connected to and used in conjunction with medical device 640, while still maintaining a relatively low endovascular device profile (e.g., relative to two separate, noncoaxial groups of conductor wires) that facilitates positioning of electrodes 670 in vasculature of a patient.

In some examples, electrodes 670 include at least twelve of electrodes 670 (e.g., sixteen of electrodes 670), although other suitable numbers of electrodes are provided in other examples (e.g., four, six, eight, twelve, fourteen, sixteen, eighteen, twenty, or more electrodes). In some examples, a number of electrodes of electrodes 670 equals a number of conductor wires of first group of conductor wires 666 (e.g., sixteen electrodes for sixteen conductor wires). However, in other examples, more than one electrode of electrodes 670 is connected to each conductor wire, such that a number of electrodes is greater than a number of conductor wires of first group of conductor wires 666 (e.g., in examples where at least some electrodes are shorted or ganged together). In some examples, the number of second group of conductor wires 668 and the number of third group of conductor wires 669 equals the number of first group of conductor wires 666. In some examples, each of second group of conductor wires 668 and third group of conductor wires 669 each include at least six conductor wires (e.g., eight conductor wires).

In some examples, as shown in the examples of FIG. 6B and FIG. 6C, medical lead 660 includes an extension element 652 extending between second lead body portion 664 and third lead body portion 665. In some examples, extension element 652 is mechanically coupled to and/or extends between an end (e.g., a proximal end) of second lead body portion 664 and an end (e.g., a distal end) of third lead body portion 665. In some examples, extension element 652 is formed from a portion of third group of conductor wires 669 that extends between second lead body portion 664 and third lead body portion 665. In some examples, extension element 652 additionally or alternatively includes an electrically insulative material that forms a lumen configured to receive third group of conductor wires 669 therethrough. In some examples, extension element 652 is relatively more flexible (e.g., has a lower flexural stiffness) as compared to one or more of second lead body portion 664 and third lead body portion 665, such that extension element 652 can deform (e.g., reversibly deform without adversely impacting the structural integrity of third group of conductor wires 669). In some examples, extension element 652 is sized, shaped, and/or otherwise configured to accommodate bending such that third lead body portion 665 can be axially offset from (e.g., but parallel to) second lead body portion 664 (e.g., as shown in the example of FIG. 6C).

In some examples, extension element 652 is sized, shaped, and/or otherwise configured to enable third lead body portion 665 to be positioned entirely proximally of second lead body portion 664 such that second lead body portion 664 and third lead body portion 665 can be positioned in sequence (e.g., in sequence along the x-axis direction according to the orthogonal x-y-z axes in FIG. 6B) and coaxial. For example, at least a portion of extension element can be coupled to a portion of third lead body portion 665 to enable third lead body portion 665, including all of second group of electrical contacts 673, to be positioned proximally of a proximal-most end of second lead body portion 664. In this way, extension element 652 can enable medical lead 660 to transform to a configuration in which at least second lead body portion 664 and third lead body portion 665 are coaxial, which may facilitate easier movement of medical lead 660 relative to and/or within a lumen a delivery catheter and/or sheath, as discussed throughout this disclosure.

In some examples, as shown in the example of FIG. 6B, extension element 652 is shaped, sized, and/or otherwise configured to enable a distal end of third lead body portion 665 to be positioned proximal of a proximal end of second lead body portion 664 by at least a distance L2 (e.g., wherein L2 is a finite distance measured along the x-axis direction according to orthogonal x-y-z axes in FIG. 6B). In other words, in some examples, in the first configuration of medical lead 660, second lead body portion 664 and third lead body portion 665 can be separated by a gap of distance L2 (e.g., the length of extension element 652). In some examples, distance L2 is sufficient (e.g., large enough) to enable a rotation of third lead body portion 665 by at least 180 degrees relative to second lead body portion 664 (e.g., such that second lead body portion 664 and third lead body portion 665 are in the relative positions shown in FIG. 6A and/or FIG. 6C). In some examples, distance L2 is sufficient (e.g., large enough) to enable third lead body portion 665 to be positioned parallel to second lead body portion 664 (e.g., in the second configuration of medical lead 660 in which second lead body portion longitudinal axis 663B and the third lead body portion longitudinal axis 663C are axially offset).

In some examples, at least a portion of extension element 652 is configured to receive at least some of the plurality of conductor wires 666, 668, 669. For example, in some examples, at least third group of conductor wires 669 are positioned along and/or within extension element 652. In some examples, extension element 652 defines a lumen configured to receive and/or house at least third group of conductor wires 669. In some examples, extension element 652 includes an electrically insulative material configured to electrically insulate at least third group of conductor wires 669 from the environment outside of (e.g., radially outside of) extension element 652.

In some examples, at least a portion of extension element 652 is configured to reversibly deform to accommodate transformation of medical lead 660 between the first configuration and the second configuration (e.g., the first configuration of medical lead 660 shown and described in connection with FIG. 6B and the second configuration of medical lead 660 as shown and described connection with FIG. 6A and/or FIG. 6C). For example, as illustrated in FIG. 6B and FIG. 6C, at least a portion of extension element 652 can be configured to flex, bend, or otherwise deform to accommodate transformation of medical lead 660 between the first configuration of medical lead 660 in which second lead body portion 664 and third lead body portion 665 are coaxial and the second configuration of medical lead 660 in which second lead body portion 664 and third lead body portion 665 are not coaxial (e.g., axially offset).

FIG. 6D illustrates a cross-sectional view of a portion of medical lead 660 illustrated in FIG. 6C, the cross section taken through a plane parallel to the x-axis and y-axis of FIG. 6C that intersecting a radial center of medical lead 660. In the example of FIG. 6D, second lead body portion 664 defines a lumen 659 configured to receive third group of conductor wires 669 therethrough. After extending through lumen 659, third group of conductor wires 669 extend to third lead body portion 665 (e.g., to electrically connect to second group of electrical contacts 673). In this way, lumen 659 enables a serial configuration of second lead body portion 664 and third lead body portion 665 that are each configured to be positioned in separate, axially offset connector portions of a medical device (e.g., medical device 640 as shown in the example of FIG. 6A).

In some examples, as shown in the FIG. of 6D, extension element 652 defines a lumen configured to receive third group of conductor wires 669 therethrough as the wires 669 extend from second lead body portion 664 to third lead body portion 665. In some examples, the lumen defined by extension element 652 is continuous with (e.g., directly adjacent to) lumen 659 of second lead body portion 664. Such continuity can ensure that third group of conductor wires 669 remain electrically insulated from the ambient environment outside of (e.g., radially outside of) each of second lead body portion 664 and extension element 652.

In some examples, second group of conductor wires 668 are configured to accommodate extension of third group of conductor wires 669 through second lead body portion 664. For example, as shown in the example of FIG. 6D, at least some wires of second group of conductor wires 668 can form a coil within the second lead body portion 664, the coil being configured to receive third group of conductor wires 669 therethrough. In some examples, the portion of third group of conductor wires 669 that extend through second lead body portion 664 and extension element 652 are uncoiled (e.g., straight wires that extend substantially parallel a longitudinal axis of each of second lead body portion 664 and extension element 652). Such a combination of an outer coil of second group of conductor wires 668 and an inner bundle of uncoiled third group of conductor wires 669 can have a relatively smaller cross-sectional profile as compared to other configurations (e.g., as compared to a dual coil configuration).

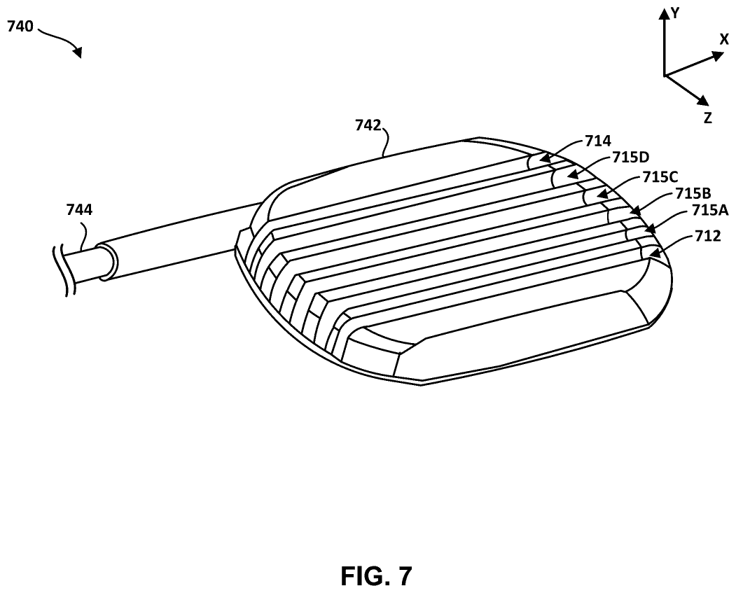

FIG. 7 illustrates a medical device 740, which may be an example of medical device 14, medical device 140, medical device 640, or any of the other medical devices described in this disclosure. In some examples, medical device 740 includes a housing 742 defining at least a first connector portion 712 and a second connector portion 714. First connector portion 712 and second connector portion 714 are examples of first connector portion 112 and second connector portion 114 of FIG. 3A. In some examples, first connector portion 712 and second connector portion 714 are configured to receive separate (e.g., physically separate) portions of a medical lead (e.g., at least a first body portion and a second body portion of a medical lead as discussed in connection with previous examples). Medical device 740 may be an example of an extension cable (e.g., Multi-lead Trailing Cable, #355531 available from Medtronic Inc.).

In some examples, housing 742 defines connector portions 715A, 715B, 715C, and 715D (collectively referred to herein as connector portions 715). In some examples, each of connector portions 715A, 715B, 715C, and 715D are configured to receive separate (e.g., physically separate) portions of a medical lead.

In some examples, one or more of each of first connector portion 712, second connector portion 714, and/or connector portions 715 include a channel that extends entirely through housing 742. In this way, portions of a medical lead can be received by housing 742 in at least two different ways (e.g., from the side of housing 742 in either the positive or negative x-axis directions according to the orthogonal x-y-z axes of FIG. 7). Such configuration of housing 742 can enable a lead having a serial configuration (e.g., as discussed with respect to FIG. 6A), to connect to multiple connector portions of medical device 740.

In some examples, medical device 740 includes a cable 744 configured to facilitate physical connection and/or electrical connection to circuitry (e.g., processing circuitry, therapy generation circuitry, sensing circuitry, and the like). In some examples, the portion of medical device 740 shown in FIG. 7 (e.g., including at least housing 742 and cable 744) is a lead extension that enables portions of a medical lead received in each of first connector portion 712, second connector portion 714, and/or connector portions 715 to electrically connect to circuitry (e.g., processing circuitry, therapy generation circuitry, sensing circuitry, and the like).

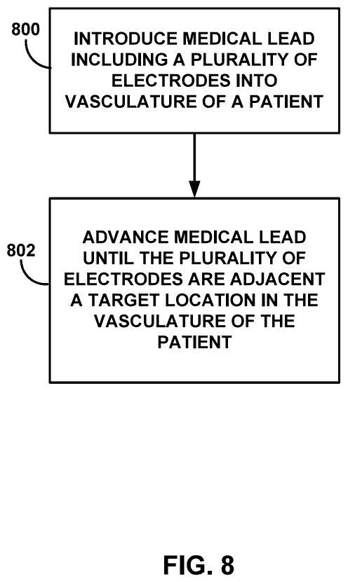

FIG. 8 is a flow diagram illustrating an example technique for using a medical device system according to the techniques of this disclosure, which may include placing a medical lead adjacent a target location in vasculature of a patient. The technique of FIG. 8 is described with respect to therapy system 10 of FIG. 1, as well as endovascular therapy system 100 of FIG. 3A, FIG. 3B, and FIG. 3C, but may be used with any of the device, systems, and/or elements of systems described in this disclosure.

In the example of FIG. 8, the technique includes introducing an endovascular device (e.g., endovascular device 16 and/or medical lead 160) into vasculature of patient 12 (800). For example, a clinician may introduce at least distal portion 150 of medical lead 160 through an access point in patient 12 including a femoral artery access point or radial artery access point. In some examples, one or more of an introducer sheath, a delivery catheter, and/or a guidewire is used to facilitate introduction of medical lead 160 into patient 12. As discussed throughout this disclosure, medical lead 160 can be configured to transform into a configuration (e.g., a first configuration as discussed in relation to the example of FIG. 3B) in which medical lead 160 is generally linear and/or has a relatively lower cross-sectional profile such that medical lead 160 moves (e.g., slides) more easily with respect to the sheath and/or delivery catheter.

In the example of FIG. 8, the technique further includes advancing medical lead 160 (e.g., while in the first configuration) through the vasculature of the patient until electrodes 170 are adjacent a target location in the vasculature of patient 12 (802). In some examples, a clinician advances medical lead 160 through vasculature of patient 12 until electrodes 170 are located within jugular vein 13 and positioned adjacent vagus nerve 21. In other examples, a clinician advances medical lead 160 through vasculature of patient 12 until electrodes 170 are located within a cranial blood vessel proximate one or more target brain structures.

In some examples, where introduction and/or advancement of medical lead 160 includes a sheath and/or delivery catheter, the technique further includes retracting the sheath and/or delivery catheter relative to (e.g., over) medical lead 160. As discussed throughout this disclosure, medical lead 160 can be configured to assume a configuration (e.g., a first configuration as discussed in relation to FIG. 3B), that enables the sheath and/or delivery catheter to move more easily relative to medical lead 160.

Once electrodes 170 are adjacent the target location (e.g., vagus nerve 21, other nerve, or one or more brain structures), the technique can include mechanically and/or electrically coupling medical lead 160 to medical device 140 (e.g., via one or more connector portions of medical device 140, such as first connector portion 112 and/or second connector portion 114). Once electrodes are electrically connected to medical device 140, the clinician can initiate (e.g., via programmer 20, or another suitable device) electrical stimulation therapy and/or sensing of one or more patient parameters by medical device 140 via electrodes 170. As discussed throughout this disclosure, medical lead 160 can be configured to transform to a configuration (e.g., a second configuration as discussed in relation to FIG. 3A and FIG. 3C) in which different portions of medical lead 160 can connect to separate, axially offset connector portions of medical device 140.

In some examples, expandable structure 190, which can be at a distal portion of medical lead 160, is configured transform from a relatively low-profile delivery configuration to a deployed configuration in a blood vessel of a patient (e.g., within jugular vein 13 of patient 12). In some examples, expandable structure 190 remains in the delivery configuration during advancement of medical lead through the vasculature.

In some examples, a clinician causes expandable structure 190 to transform to the deployed (e.g., expanded) configuration once electrodes 170 are adjacent the target site. In the deployed configuration of expandable structure 190, one or more of electrodes 170 can be positioned into apposition with the vessel wall (e.g., the vessel wall of jugular vein 13).

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

This disclosure includes the following non-limiting examples.
Example 1: An endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires splits to form the second group of conductor wires and the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.
Example 2: The endovascular device of example 1, wherein in the first configuration, the second lead body portion and the third lead body portion are configured to enable a delivery catheter to be retracted over each the second lead body portion and the third lead body portion in sequence and, wherein the second lead body portion and the third lead body portion are configured to pass through a lumen of the delivery catheter.
Example 3: The endovascular device of any of examples 1 and 2, wherein in the first configuration, the second lead body portion longitudinal axis and the third lead body portion longitudinal axis are coaxial with the first lead body portion longitudinal axis.
Example 4: The endovascular device of any of examples 1 through 3, wherein in the second configuration, the second lead body portion is configured to position the second group of conductor wires to electrically connect to the medical device via the first connector portion and the third lead body portion is configured to position and the third group of conductor wires to electrically connect to the medical device via the second connector portion.
Example 5: The endovascular device of any of examples 1 through 4, wherein the second lead body portion includes a first group of electrical contacts along the second lead body portion longitudinal axis, each electrical contact of the first group of electrical contacts being electrically connected to at least one wire of the second group of conductor wires, wherein the third lead body portion includes a second group of electrical contacts along the third lead body portion longitudinal axis, each electrical contact of the second group of electrical contacts being electrically connected to at least one wire of the third group of conductor wires, wherein in the first configuration, all electrical contacts of the second group of electrical contacts are proximal to all electrical contacts of the first group of electrical contacts.
Example 6: The endovascular device of any of examples 1 through 5, further comprising a connection body positioned at a junction, the junction separating the first lead body portion from the second lead body portion and the third lead body portion, wherein the first group of conductor wires splits to form the second group of conductor wires and the third group of conductor wires at the junction.
Example 7: The endovascular device of example 6, wherein the connection body includes a first connection body portion and a second connection body portion, the first connection body portion configured to mechanically couple to the lead body and the second connection body portion configured to extend parallel to and along at least a portion of the lead body.
Example 8: The endovascular device of example 7, wherein at least the third group of conductor wires is positioned within second connection body portion of the connection body.
Example 9: The endovascular device of any of examples 6 through 8, wherein the connection body is configured to reversibly deform to accommodate transformation of the endovascular device between the first configuration and the second configuration.
Example 10: The endovascular device of any of examples 1 through 9, wherein at least a first subset of the first group of conductor wires forms a first coil and a second subset of the first group of conductor wires is positioned within the first coil, wherein the second group of conductor wires forms a second coil, and wherein a section of uncoiled conductor wires separates the first coil and the second coil to enable the second subset of the first group of conductor wires to exit the first coil.
Example 11: The endovascular device of any of examples 1 through 9, wherein at least a first subset of the first group of conductor wires forms a first coil defining a first pitch, wherein a second subset of the first group of conductor wires is positioned within the first coil, wherein the second group of conductor wires forms a second coil having a second pitch, wherein a section of coiled wires separates the first coil and the second coil, and wherein the section of coiled conductor wires that separates the first coil and the second coil defines a third pitch greater than the first pitch and the second pitch to enable the second subset of the first group of conductor wires to exit the first coil.
Example 12: The endovascular device of any of examples 1 through 11, wherein the second group of conductor wires and the third group of conductor wires each include at least six conductor wires.
Example 13: The endovascular device of any of examples 1 through 12, wherein the plurality of electrodes includes at least twelve electrodes.
Example 14: The endovascular device of any of examples 1 through 13, further including an expandable structure at a distal portion of the first lead body portion, the expandable structure configured to transform from a relatively low-profile delivery configuration to a deployed configuration in a blood vessel of a patient, wherein the plurality of electrodes are disposed on the expandable structure.
Example 15: A medical system including the endovascular device of any of examples 1 through 14; and the medical device, the medical device including at least the first connector portion and the second connector portion, the first connector portion configured to receive the second lead body portion and the second connector portion configured to receive the third lead body portion.
Example 16: An endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires transitions to form the second group of conductor wires and the third group of conductor wires, wherein the third group of conductor wires is configured to extend through the second lead body portion to the third lead body portion, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.
Example 17: The endovascular device of example 16, wherein in the first configuration, the second lead body portion and the third lead body portion are configured to enable a delivery catheter to be retracted over the second lead body portion and the third lead body portion in sequence and, wherein the second lead body portion and the third lead body portion are configured to pass through a lumen of the delivery catheter.
Example 18: The endovascular device of any of examples 16 and 17, wherein in the first configuration, the second lead body portion longitudinal axis and the third lead body portion longitudinal axis are coaxial with the first lead body portion longitudinal axis.
Example 19: The endovascular device of any of examples 16 through 18, wherein in the second configuration, the second lead body portion is configured to position the second group of conductor wires to electrically connect to the medical device via the first connector portion and the third lead body portion is configured to position the third group of conductor wires to electrically connect to the medical device via the second connector portion.
Example 20: The endovascular device of any of examples 16 through 19, wherein the second lead body portion includes a first group of electrical contacts along the second lead body portion longitudinal axis, each electrical contact of the first group of electrical contacts being electrically connected to at least one wire of the second group of conductor wires, wherein the third lead body portion includes a second group of electrical contacts along the third lead body portion longitudinal axis, each electrical contact of the second group of electrical contacts being electrically connected to at least one wire of the third group of conductor wires, wherein in the first configuration, all electrical contacts of the second group of electrical contacts are proximal to all electrical contacts of the first group of electrical contacts.
Example 21: The endovascular device of any of examples 16 through 20, wherein the second lead body portion defines a lumen, the lumen configured to receive the third group of conductor wires therethrough.
Example 22: The endovascular device of any of examples 16 through 21, wherein the second group of conductor wires forms a coil within the second lead body portion, the coil configured to receive the third group of conductor wires therethrough.
Example 23: The endovascular device of any of examples 16 through 22, further comprising an extension element between the second lead body portion and the third lead body portion, the extension element configured to receive the third group of conductor wires therethrough.
Example 24: The endovascular device of example 23, wherein the extension element is configured to reversibly deform to accommodate transformation between the first configuration and the second configuration.
Example 25: The endovascular device of any of examples 23 and 24, wherein the extension element defines a length sufficient to enable the third lead body portion to be parallel to the second lead body portion in the second configuration in which the second lead body portion longitudinal axis and the third lead body portion longitudinal axis are axially offset.
Example 26: The endovascular device of any of examples 23 through 25, wherein the extension element extends between a proximal end of the second lead body portion and a distal end of the third lead body portion.
Example 27: The endovascular device of any of examples 16 through 26, wherein the second group of conductor wires and the third group of conductor wires each include at least six conductor wires.
Example 28: The endovascular device of any of examples 16 through 27, wherein the plurality of electrodes includes at least twelve electrodes.
Example 29: The endovascular device of any of examples 16 through 28, further including an expandable structure at a distal portion of the first lead body portion, the expandable structure configured to transform from a relatively low-profile delivery configuration to a deployed configuration in a blood vessel of a patient, wherein the plurality of electrodes are disposed on the expandable structure.
Example 30: A medical system including the endovascular device of any of examples 16 through 29; and the medical device, the medical device including at least the first connector portion and the second connector portion, the first connector portion configured to receive the second lead body portion and the second connector portion configured to receive the third lead body portion.
Example 31: An endovascular device includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset.
Example 32: A method includes introducing a medical lead into vasculature of a patient, the medical lead includes a plurality of conductor wires including at least a first group of conductor wires, a second group of conductor wires, and a third group of conductor wires; a lead body including a first lead body portion, a second lead body portion, and a third lead body portion; and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires splits to form the second group of conductor wires and the third group of conductor wires, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, an wherein in a second configuration, the second lead body portion longitudinal axis and the third lead body portion longitudinal axis are axially offset; and advancing the medical lead until the plurality of electrodes are adjacent a target location in the vasculature of the patient.
Example 33: A method includes introducing a medical lead into vasculature of a patient, the medical lead includes a plurality of conductor wires including at least a first group of conductor wires, a second group of conductor wires, and a third group of conductor wires, a lead body including a first lead body portion, a second lead body portion, and a third lead body portion, and a plurality of electrodes, each electrode of the plurality of electrodes electrically connected to at least one conductor wire of the first group of conductor wires, wherein the first lead body portion defines a first lead body portion longitudinal axis, the second lead body portion defines a second lead body portion longitudinal axis, and the third lead body portion defines a third lead body portion longitudinal axis, wherein the first lead body portion includes the first group of conductor wires, the second lead body portion includes the second group of conductor wires, and the third lead body portion includes the third group of conductor wires, wherein the first group of conductor wires transitions to form the second group of conductor wires and the third group of conductor wires, wherein the third group of conductor wires is configured to extend through the second lead body portion to the third lead body portion, wherein the second lead body portion is configured to be received in a first connector portion of a medical device to electrically connect the second group of conductor wires to a first set of electrical contacts of the medical device, wherein the third lead body portion is configured to be received in a second connector portion of the medical device to electrically connect the third group of conductor wires to a second set of electrical contacts of the medical device, wherein in a first configuration, the second lead body portion longitudinal axis and the third lead body portion longitudinal axis are coaxial, and wherein in a second configuration, the second lead body portion longitudinal axis and the third lead body portion longitudinal axis are axially offset; and advancing the medical lead until the plurality of electrodes are adjacent a target location in the vasculature of the patient.

Various examples of the disclosure have been described. Any combination of the described systems, operations, or functions is contemplated. These and other examples are within the scope of the following claims.

What is claimed is:

1. An endovascular device comprising:
a plurality of conductor wires including at least a first group of conductor wires, a second group of conductor wires, and a third group of conductor wires;
a lead body including a first lead body portion, a second lead body portion, and a third lead body portion; and
a plurality of electrodes, each electrode of the plurality of electrodes electrically connected to at least one conductor wire of the first group of conductor wires,
wherein the first lead body portion defines a first lead body portion longitudinal axis, the second lead body portion defines a second lead body portion longitudinal axis, and the third lead body portion defines a third lead body portion longitudinal axis,
wherein the first lead body portion includes the first group of conductor wires, the second lead body portion includes the second group of conductor wires, and the third lead body portion includes the third group of conductor wires,
wherein the first group of conductor wires splits to form the second group of conductor wires and the third group of conductor wires,
wherein the second lead body portion is configured to be received in a first connector portion of a medical device to electrically connect the second group of conductor wires to a first set of electrical contacts of the medical device,
wherein the third lead body portion is configured to be received in a second connector portion of the medical device to electrically connect the third group of conductor wires to a second set of electrical contacts of the medical device,
wherein in a first configuration, the second lead body portion longitudinal axis and the third lead body portion longitudinal axis are coaxial, and
wherein in a second configuration, the second lead body portion longitudinal axis and the third lead body portion longitudinal axis are axially offset.

2. The endovascular device of claim 1,
wherein in the first configuration, the second lead body portion and the third lead body portion are configured to enable a delivery catheter to be retracted over each the second lead body portion and the third lead body portion in sequence and,
wherein the second lead body portion and the third lead body portion are configured to pass through a lumen of the delivery catheter.

3. The endovascular device of claim 1, wherein in the first configuration, the second lead body portion longitudinal axis and the third lead body portion longitudinal axis are coaxial with the first lead body portion longitudinal axis.

4. The endovascular device of claim 1, wherein in the second configuration, the second lead body portion is configured to position the second group of conductor wires to electrically connect to the medical device via the first connector portion and the third lead body portion is configured to position and the third group of conductor wires to electrically connect to the medical device via the second connector portion.

5. The endovascular device of claim 1
wherein the second lead body portion includes a first group of electrical contacts along the second lead body portion longitudinal axis, each electrical contact of the first group of electrical contacts being electrically connected to at least one wire of the second group of conductor wires,
wherein the third lead body portion includes a second group of electrical contacts along the third lead body portion longitudinal axis, each electrical contact of the second group of electrical contacts being electrically connected to at least one wire of the third group of conductor wires,
wherein in the first configuration, all electrical contacts of the second group of electrical contacts are proximal to all electrical contacts of the first group of electrical contacts.

6. The endovascular device of claim 1, further comprising a connection body positioned at a junction, the junction separating the first lead body portion from the second lead body portion and the third lead body portion, wherein the first group of conductor wires splits to form the second group of conductor wires and the third group of conductor wires at the junction, wherein the connection body includes a first connection body portion and a second connection body portion, the first connection body portion configured to mechanically couple to the lead body and the second connection body portion configured to extend parallel to and along at least a portion of the lead body, wherein at least the third group of conductor wires is positioned within second connection body portion of the connection body, and wherein the connection body is configured to reversibly deform to accommodate transformation of the endovascular device between the first configuration and the second configuration.

7. The endovascular device of claim 1,
wherein at least a first subset of the first group of conductor wires forms a first coil and a second subset of the first group of conductor wires is positioned within the first coil,
wherein the second group of conductor wires forms a second coil, and
wherein a section of uncoiled conductor wires separates the first coil and the second coil to enable the second subset of the first group of conductor wires to exit the first coil.

8. The endovascular device of claim 1,
wherein at least a first subset of the first group of conductor wires forms a first coil defining a first pitch,
wherein a second subset of the first group of conductor wires is positioned within the first coil,
wherein the second group of conductor wires forms a second coil having a second pitch,
wherein a section of coiled wires separates the first coil and the second coil, and
wherein the section of coiled conductor wires that separates the first coil and the second coil defines a third pitch greater than the first pitch and the second pitch to enable the second subset of the first group of conductor wires to exit the first coil.

9. The endovascular device of claim 1, wherein the second group of conductor wires and the third group of conductor wires each include at least six conductor wires.

10. The endovascular device of claim 1, wherein the plurality of electrodes includes at least twelve electrodes.

11. The endovascular device of claim 1, further including an expandable structure at a distal portion of the first lead body portion, the expandable structure configured to transform from a relatively low-profile delivery configuration to a deployed configuration in a blood vessel of a patient, wherein the plurality of electrodes are disposed on the expandable structure.

12. A medical system comprising,
the endovascular device of claim 1; and
the medical device, the medical device including at least the first connector portion and the second connector portion, the first connector portion configured to receive the second lead body portion and the second connector portion configured to receive the third lead body portion.

13. An endovascular device comprising:
a plurality of conductor wires including at least a first group of conductor wires, a second group of conductor wires, and a third group of conductor wires;
a lead body including a first lead body portion, a second lead body portion, and a third lead body portion; and
a plurality of electrodes, each electrode of the plurality of electrodes electrically connected to at least one conductor wire of the first group of conductor wires,
wherein the first lead body portion defines a first lead body portion longitudinal axis, the second lead body portion defines a second lead body portion longitudinal axis, and the third lead body portion defines a third lead body portion longitudinal axis,
wherein the first lead body portion includes the first group of conductor wires, the second lead body portion includes the second group of conductor wires, and the third lead body portion includes the third group of conductor wires,
wherein the first group of conductor wires transitions to form the second group of conductor wires and the third group of conductor wires,
wherein the third group of conductor wires is configured to extend through the second lead body portion to the third lead body portion,
wherein the second lead body portion is configured to be received in a first connector portion of a medical device to electrically connect the second group of conductor wires to a first set of electrical contacts of the medical device,
wherein the third lead body portion is configured to be received in a second connector portion of the medical device to electrically connect the third group of conductor wires to a second set of electrical contacts of the medical device,
wherein in a first configuration, the second lead body portion longitudinal axis and the third lead body portion longitudinal axis are coaxial, and
wherein in a second configuration, the second lead body portion longitudinal axis and the third lead body portion longitudinal axis are axially offset.

14. The endovascular device of claim 13,
wherein in the first configuration, the second lead body portion and the third lead body portion are configured to enable a delivery catheter to be retracted over the second lead body portion and the third lead body portion in sequence and,
wherein the second lead body portion and the third lead body portion are configured to pass through a lumen of the delivery catheter.

15. The endovascular device of claim 14, wherein in the first configuration, the second lead body portion longitudinal axis and the third lead body portion longitudinal axis are coaxial with the first lead body portion longitudinal axis.

16. The endovascular device of claim 15, wherein in the second configuration, the second lead body portion is configured to position the second group of conductor wires to electrically connect to the medical device via the first connector portion and the third lead body portion is configured to position the third group of conductor wires to electrically connect to the medical device via the second connector portion.

17. The endovascular device of claim 16,
wherein the second lead body portion includes a first group of electrical contacts along the second lead body portion longitudinal axis, each electrical contact of the first group of electrical contacts being electrically connected to at least one wire of the second group of conductor wires,
wherein the third lead body portion includes a second group of electrical contacts along the third lead body portion longitudinal axis, each electrical contact of the second group of electrical contacts being electrically connected to at least one wire of the third group of conductor wires,
wherein in the first configuration, all electrical contacts of the second group of electrical contacts are proximal to all electrical contacts of the first group of electrical contacts.

18. The endovascular device of claim 17,
wherein the second lead body portion defines a lumen, the lumen configured to receive the third group of conductor wires therethrough.

19. The endovascular device of any claim 18,
wherein the second group of conductor wires forms a coil within the second lead body portion, the coil configured to receive the third group of conductor wires therethrough.

20. The endovascular device of claim 19, further comprising an extension element between the second lead body portion and the third lead body portion, the extension element configured to receive the third group of conductor wires therethrough, and wherein the extension element is configured to reversibly deform to accommodate transformation between the first configuration and the second configuration.
